(19) United States
(12) Patent Application Publication
(10) Pub. No.: US 2022/0411397 A1
(43) Pub. Date: Dec. 29, 2022

(54) ANALOGUES OF N-ACYL-HOMOSERINE LACTONES AND PHARMACEUTICAL COMPOSITION COMPRISING SAME

(71) Applicants: SORBONNE UNIVERSITE, PARIS (FR); INSTITUT NATIONAL DE LA SANTE ET DE LA RECHERCHE MEDICALE (INSERM), PARIS (FR); ASSISTANCE PUBLIQUE- HOPITAUX DE PARIS, PARIS (FR); CENTRE NATIONAL DE LARECHERCHE SCIENTIFIQUE, PARIS (FR); ECOLE NORMALE SUPERIEURE, PARIS CEDEX 05 (FR)

(72) Inventors: Philippe SEKSIK, PARIS (FR); Jean-Pierre GRILL, SAINT MAUR DES FOSSES (FR); Jean-Maurice MALLET, VITRY SUR SEINE (FR); Dominique RAINTEAU, CRETEIL (FR); Cecilia LANDMAN, PARIS (FR); Agathe PEYROTTES, PARIS (FR); Loic BROT, PARIS (FR)

(21) Appl. No.: 17/770,502

(22) PCT Filed: Oct. 23, 2020
(86) PCT No.: PCT/EP2020/079913

§ 371 Date: Apr. 20, 2022

(30) Foreign Application Priority Data
Oct. 23, 2019 (FR) FR1911861

(51) Int. Cl. C07D 333/32; C07C 235/74

(52) U.S. Cl. CPC C07D 333/32; C07C 235/74

(57) ABSTRACT

The present invention relates to analogues of N-acyl homoserine lactones (AHLs) and pharmaceutical compositions comprising same. The invention also relates to their use in the treatment of inflammatory diseases of the epithelium.

The invention relates to analogues of N-acyl-homoserine lactones (AHLs) and pharmaceutical compositions comprising same.

It also concerns their use in the treatment of inflammatory diseases of the epithelium.

The inflammatory diseases of the epithelium affect both the digestive tract and the skin.

Indeed, these organs are in direct contact with the external medium and their microbiome is specific.

The inflammatory skin diseases comprise the psoriasis.

The chronic inflammatory bowel diseases (IBD) are represented by two main diseases: the Crohn's disease (CD) and the ulcerative colitis (UC) characterised by a chronic inflammation of the intestinal mucosa leading to damage of the intestine and an increased risk of intestinal cancer (multiplied by 4 compared to the general population for the colon and multiplied by 40 for the small intestine).

The socio-economic consequences are important because the IBDs affect young subjects and their incidence is relatively high in industrialized countries (8 to 15 per 100,000 inhabitants/year).

The long-term risk is estimated at 1% in Europe and both CD and UC remain incurable diseases.

Recent advances in the treatments of the IBDs including the expansion of biologic agents have resulted in rapid clinical remission and an improved quality of life for many IBD patients.

However, these potent immunosuppressive therapies are not always effective, are costly and potentially induce serious side effects.

There is therefore a need for more physiological approaches to induce and maintain the remissions with a limited toxicity and a high cost effectiveness.

The CD can a priori affect all the segments of the digestive tract, but more frequently affects the ileum, the colon and the anus. The disease presents a transmural inflammation, i.e. affecting the different layers of the digestive wall, which can cause severe complications in patients, such as stenosis and fistulae.

The UC affects the rectum and the terminal part of the colon. The resulting inflammation is usually limited to the intestinal mucosa and submucosa.

The pathophysiology of the IBD is complex and still not well understood, but it clearly involves multiple factors, including environmental causes, a genetic predisposition, immune disturbances, a defect in the barrier function, and an imbalance, called dysbiosis, of the intestinal flora: the microbiota.

The intestinal microbiota plays an important role in controlling the inflammation and in regulating the barrier function. Several mechanisms of barrier reinforcement have been described, such as the stimulation of the anti-microbial peptide or mucus secretion, but few works have analysed the influence of the commensal microbiota on the permeability of tight junctions, which are the key players in the control of the paracellular permeability.

The quorum sensing (QS) is a mode of communication between the individuals in a bacterial colony, based on the secretion of small diffusible molecules into the surrounding environment, which act as chemical messengers and are referred to as auto-inducers (AI).

The quorum sensing is based on the density of bacteria present in the medium and the concentration of signal molecules in their immediate environment. The quantity of molecules in the medium is a direct reflection of the state of the colony: when the number of individuals is low, the concentration of auto-inducers is also low; but when the cell density increases, the concentration of molecules increases in turn until it crosses a threshold beyond which one or more bacterial phenotypes are modified. This threshold is called quorum. The term sensing refers to the ability of the bacteria to detect molecules in the medium via appropriate receptors, hence the name auto-inducers because the molecules are both secreted and detected.

The quorum sensing involving the N-acyl-homoserine lactones (AHLs) is a mode of inter-bacterial communication described in the Gram-negative bacteria in many ecosystems.

These molecules are also capable of exerting effects on the host cells, in an “inter-kingdom dialogue”. However, the presence of molecules of the AHLs type in the intestinal ecosystem has been poorly described until now.

The intestinal microbiota includes all the micro-organisms (bacteria, yeasts, archaea and viruses) present in our digestive tract, including 10¹⁴ bacteria (about 10 times the number of human cells, although this ratio is subject to discussion) divided into a thousand species. The relationship between the host and its intestinal microbiota is symbiotic, as it mutually benefits both parties. The host provides nutrients via its food bolus, while the microbiota performs many functions of a physiological nature (metabolism of carbohydrates and lipids, transformation of bile acids etc.), immune (“education” of the immune system), but also ecological, because the occupation of the intestinal space by a commensal flora prevents the colonization by pathogenic species.

Although the microbiota of an adult individual is unique, major common characteristics have been identified to describe a healthy microbiota in a normobiosis state. The dominant microbiota or core microbiome consists of 4 phyla: Firmicutes and Bacteroidetes are strongly represented, and to a lesser extent the phyla Actinobacteria and Proteobacteria.

In certain situations, an imbalance in the representation of the bacterial populations normally present can appear: this is the dysbiosis. It has thus been shown that there is a state of dysbiosis in the IBD, which is certainly a key element in the development of these pathologies, although these mechanisms of development are not yet elucidated. This dysbiosis is characterised by a loss of diversity with changes in the microbial composition (decreased Bacteroidetes and Firmicutes with in particular a significant loss of the genus Clostridia, increase of the Gamma proteobacteria and appearance of new groups such as the A/EC and Fusobacterium) and in the functions of the microbiota (decreased amino acid metabolism and biosynthesis of SCFAs and butyrate, increase of oxidative stress etc.).

As described above, the QS is involved in the bacterial interspecies communication, but can also be involved in the host-microbiota (inter-kingdom) relationships. It has been described in marine ecosystems and various rhizospheres, but also in some gastrointestinal pathogens such as Yersinia. The latter is capable of secreting at least eight different AHLs and has two LuxI/LuxR homologous systems, which make it sensitive to the AHLs produced by other bacterial populations. Other species have been described as possessing the SdiA receptor (a LuxR homologous receptor not associated with an AHL synthase, and thus present in species capable of sensing the AHLs without synthesizing them themselves).

More and more studies are investigating the impact of the AHLs, and particularly of the 3-oxo-C₁₂ HSL produced by the pathogen Pseudomonas aeruginosa, on the human host cells.

It has been shown that this molecule can modulate the immune response of the host by acting as a virulence factor in itself and lead to an inflammatory response by induction of immune cells and pro-inflammatory cytokines through the expression of NF-kappaB. The entry of the AHL into the eukaryotic cells was demonstrated in 2007 by Ritchie et al. (Ritchie A J, Jansson A, Stallberg J, Nilsson P, Lysaght P, Cooley M A, The Pseudomonas aeruginosa Quorum-Sensing Molecule N-3-(Oxododecanoyl)-L-Homoserine Lactone Inhibits T-Cell Differentiation and Cytokine Production by a Mechanism Involving an Early Step in T-Cell Activation, Infect Immun. 2005;73: 1648-1655, doi:10.1128/IAI.73.3.1648-1655,2005).

The attention has thus turned to the detection of molecules of the QS in the intestinal microbiota: the synthesis of the molecule of the self-induced QS of the type 2 (AI-2) has been reported in nine commensal species and in the stools of healthy subjects, but the studies on the AHLs (AI-1) remain rarer. The presence of AHLs in the stools of newborns was demonstrated by bioluminescence.

The inventors were able to identify several AHLs in the intestinal ecosystem, one of which is lost in the IBD patients during the inflammatory flare, a 3-oxo-C₁₂:2-HSL of the following formula B:

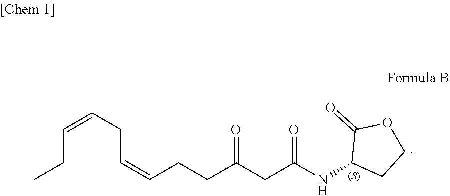

They showed that this AHL was associated with a healthy microbiota.

To date, two “natural” AHL, the 3-oxo-C₁₂-HSL of the following formula A:

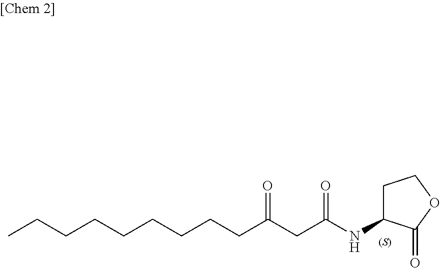

and the 3-oxo-C₁₂:2-HSL of formula B (activities disclosed in Inter-kingdom effect on epithelial cells of the N-Acyl homoserine lactone 3-oxo-C₁₂:2, a major quorum-sensing molecule from gut microbiota, Landman C, Grill J P, Mallet J M, Marteau P, Humbert L, Le Balc'h E, Maubert M A, Perez K, Chaara W, Brot L, Beaugerie L, Sokol H, Thenet S, Rainteau D, Seksik P, Quevrain E; Saint Antoine IBD Network, PLoS One, 20129;13(8):e0202587 doi:10.1371/journal.pone.0202587. eCollection 2018) are known and described as molecules with an anti-inflammatory effect and an action on the tight junctions.

These two molecules (the 3-oxo-C₁₂-HSL produced by a pathogen and the 3-oxo-C₁₂:2-HSL associated with a healthy microbiota) both act on the eukaryotic cells of the intestinal ecosystem.

However, the “natural” AHLs are molecules with poor stability. The degradation of the AHLs can lead to two different by-products: on the one hand the hydrolysis of the lactone head produces an open form of the AHL with a new alcohol function and a carboxylic acid (this molecule will be named here 3-oxo-C₁₂-HS); on the other hand, a rearrangement of the molecule can give a new molecule referred to as tetramic acid. The transformation of the AHLs into the open form can take place by spontaneous hydrolysis in aqueous medium, or be catalysed by enzymes secreted by the mammalian intestinal epithelium, and in particular the Paraoxonases (PON1, 2 and 3).

In addition to the question of the stability of the AHLs, the recognition of these molecules by bacterial receptors is problematic. It should be kept in mind that these are primarily compounds secreted by bacteria for bacteria, and that they therefore have receptors for AHLs whose activation leads to a cascade of reactions resulting in the modification of a phenotype. Among others, in P. aeruginosa, the activation of the receptor to the LasR AHL, whose natural ligand is the 3-oxo-C₁₂-HSL, leads to the downstream biofilm formation and an increased secretion of several virulence factors, such as the pyocyanin.

This activation of the bacterial receptors is problematic because it prevents the direct use of AHL against the inflammation, in particular in the IBD. The risks of activating the pathogenicity of certain bacterial strains are undesirable effects that are far too important.

In this context, the invention aims to provide bio-inspired molecules of natural AHLs (analogues of the natural AHLs) whose structural modifications aim:
to increase their stability,
to limit their toxicity,
to allow their direct use as therapeutic molecules,
to modulate their biological activity,
to allow studying their impact on the host,
to exploit their properties for therapeutic purposes.

To this end, the invention provides a compound having the following general formula I:

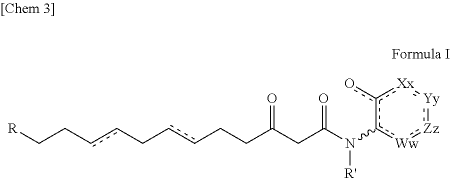

wherein
X, Y, Z and W are independently of each other a carbon atom or a heteroatom selected from S, N and O, provided that X is different from O,
X, Y, Z and W are independently of each other optionally substituted with a halogen selected from Cl, F, Br, and I, or a linear or branched C₁ to C₄ alkyl group,
x, y, z, and w, independently of each other, are 0 or 1, provided that 3≤x+y+z+w≤4,
R represents H or a linear or branched C₁ to C₄ alkyl group, or a hydroxyl group (OH) or an azido group (N₃),
R′ represents H or a linear or branched C₁ to C₄ alkyl group
[symbol] represents a single or double bond (-cis or trans)

for use in the treatment of an inflammatory disease of the epithelium.

The inflammatory disease of the epithelium is more specifically an inflammatory intestinal disease or the psoriasis.

Preferably, the compound of formula I is selected from the group consisting of:
the (D/L)-3oxoC₁₂ aminothiolactone ((D/L)-3oxoC₁₂-HTL) of the following formula I-1:

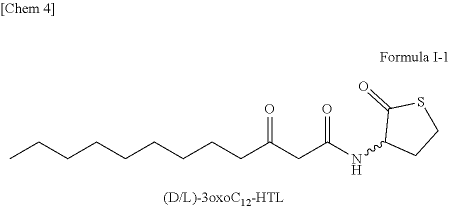 [SMILES: CCCCCCCCCC(=O)CC(=O)NC1CCSC1=O]

the (S,S)-3-oxo C₁₂-aminocyclohexanol (S,S)-3-oxo C₁₂-ACH) of the following formula I-2:

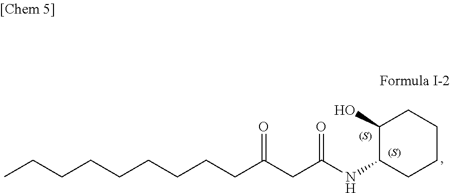

the (S)-3-oxo C₁₂ aminothiolactone ((S)-3oxoC₁₂-HTL) of the following formula I-3:

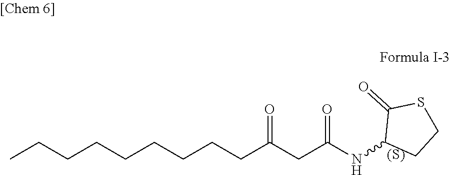

the (R,S)-3-oxo C₁₂-aminocyclohexanol of the following formula I-4:

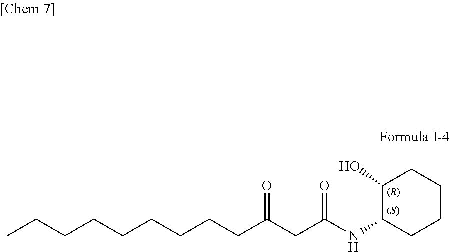

the 3-oxo C₁₂-aminocyclohexanol of the following formula I-5:

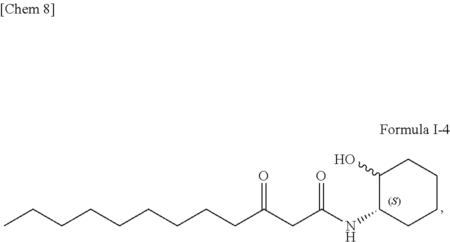

the 3-oxo C₁₂-aminochlorophenol of the following formula I-6:

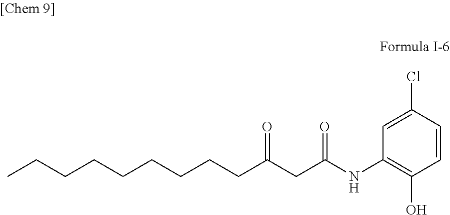 [SMILES: CCCCCCCCCC(=O)CC(=O)Nc1cc(Cl)ccc1O]

The invention also provides a pharmaceutical composition comprising:
at least one compound having the following general formula I:

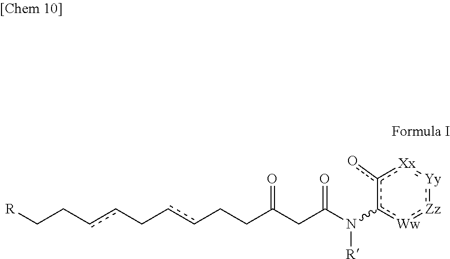

wherein:
X, Y, Z and W are independently of each other a carbon atom or a heteroatom selected from S, N and O, provided that X is different from O,
X, Y, Z and W are independently of each other optionally substituted with a halogen selected from Cl, F, Br, and I, or a linear or branched C₁ to C₄ alkyl group,
x, y, z, and w, independently of each other, are 0 or 1, provided that 3≤x+y+z+w≤4,
R represents H or a linear or branched C₁ to C₄ alkyl group, or a hydroxyl group (OH) or an azido group (N₃),
R′ represents H or a linear or branched C₁ to C₄ alkyl group
[symbol] represents a single or double bond (-cis or trans), and
at least one pharmaceutically acceptable excipient.

Preferably, in the pharmaceutical composition of the invention, the at least one compound of formula I is selected from the group consisting of:
the (D/L)-3oxoC₁₂ aminothiolactone ((D/L)-3-oxo-C₁₂-HTL) of the following formula I-1:

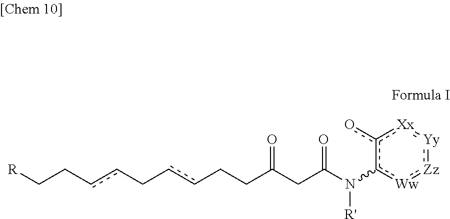

the (S,S)-3-oxo C₁₂ aminocyclohexanol (S,S)-3-oxo C₁₂-ACH) of the following formula I-2:

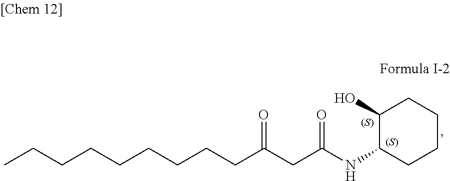

the (S)-3-oxo C₁₂ aminothiolactone ((S)-3oxoC₁₂-HTL) of the following formula I-3:

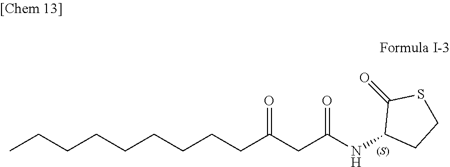

the (R,S)-3-oxo C₁₂-aminocyclohexanol of the following formula I-4:

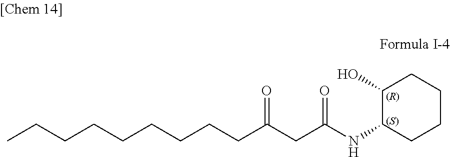

the 3-oxo C₁₂-aminocyclohexanol of the following formula I-5:

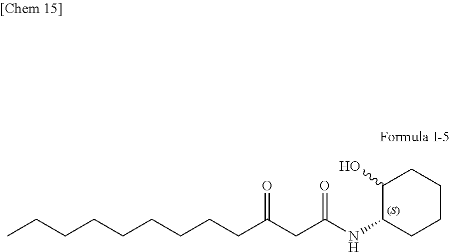

3-oxo C₁₂-aminochlorophenol of the following formula I-6:

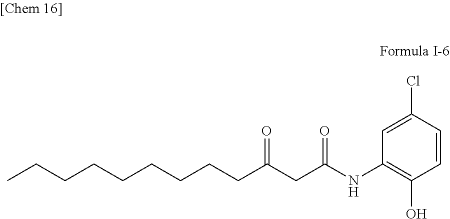 [SMILES: CCCCCCCCCC(=O)CC(=O)Nc1cc(Cl)ccc1O]

The pharmaceutical composition according to the invention is preferably for use in the treatment of an inflammatory disease of the epithelium, more particularly an inflammatory disease of the intestine or the psoriasis.

The invention will be better understood and other advantages and characteristics thereof will become clearer upon reading the following explanatory description, which is made in connection with the attached figures in which:

### FIGURES

FIG. 1 shows in bar graph form the results of stimulation of Caco-2/TC7 cells by the IL-1β in the presence of (D,L)-3oxo C₁₂ aminothiolactone of formula I-1 used in the invention,

FIG. 2 shows the activation curves of the LasR receptor on bacterial reporter strain in relation to the natural molecules C4-HSL and 3-oxo-C₁₂-HSL and to the molecule of formula I-1 used in the invention,

FIG. 3 shows in bar graph form the results of stimulation of Caco-2/TC7 cells by the IL-1β in the presence of (S,S)-3-oxo C₁₂ aminocyclohexanol (S,S)-3-oxo C₁₂-ACH) of formula I-2 used in the invention,

FIG. 4 represents the inhibition curve of the IL-8 secretion by human keratinocytes stimulated by IL-17 and TNF-α in the presence of increasing doses of (S,S) 3-oxo C₁₂ aminocyclohexanol (S,S) 3-oxo C₁₂-ACH) of formula I-2 used in the invention,

FIG. 5 shows the inhibition curve of the IL-2 secretion by human lymphocytes stimulated by CD2, CD3 and CD28, in the presence of increasing doses of (S,S)-3-oxo C₁₂-aminocyclohexanol (S,S)-3-oxo C₁₂-ACH) of formula I-2 used in the invention,

FIG. 6 shows the activation curves of the LasR receptor on the bacterial reporter strain in relation to the natural 3-oxo-C₁₂HSL molecule and to the molecule of formula I-2 used in the invention,

FIG. 7 shows in bar graph form the results of stimulation of Caco-2/TC7 cells by the IL-1β in the presence of (S)-3-oxo C₁₂ aminothiolactone ((S)-3oxoC₁₂-HTL) of formula I-3 used in the invention,

FIG. 8 represents in bar graph form the results of stimulation of Raw 264.7 murine cells by the IFN-γ/LPS combination in the presence of (S,S)-3-oxo C₁₂ aminocyclohexanol ((S,S)-3 oxo C₁₂-ACH) of formula I-2 used in the invention,

FIG. 9 shows in bar graph form the results of stimulation of Caco-2/TC7 cells by the IL-1β in the presence of (R,S)-3-oxo C₁₂ aminocyclohexanol (R,S)-3-oxo C₁₂-ACH) of formula I-4 used in the invention,

FIG. 10 represents in bar graph form the results of stimulation of Raw 264.7 murine cells by the IFN-γ/LPS combination in the presence of the (R,S) 3-oxo C₁₂-ACH) of formula I-4 used in the invention,

FIG. 11 shows the activation curves of the LasR receptor on the bacterial reporter strain in relation to the natural molecule 3-oxo-C₁₂-HSL and to the (R,S)-3-oxo C₁₂-aminocyclohexanol of formula I-4 used in the invention,

FIG. 12 shows in bar graph form the results of stimulation of Caco-2/TC7 cells by the IL-1β in the presence of the 3-oxo C₁₂-aminochlorophenol of formula I-6 used in the invention,

FIG. 13 represents in bar graph form the results of stimulation of Raw 264.7 murine cells by the IFN-γ/LPS combination in the presence of the 3-oxo C₁₂-aminochlorophenol of formula I-6 used in the invention, and

FIG. 14 shows the activation curves of the LasR receptor on the bacterial reporter strain in relation to the natural molecule 3-oxo-C₁₂-HSL and to the 3-oxo C₁₂-aminochlorophenol of formula I-6 used in the invention.

FIG. 15 represents as heat map form the secretion of 23 cytokines by Raw264.7 murine cells under stimulated conditions (LPS 10 ng/mL; IFN-γ 20 U/mL) (normalized to control),

FIGS. 16A and 16B show in histogram form:

At the top, the amount of TNF∝ secreted by Raw264.7.7 murine cells stimulated by LPS and interferonγ in the presence of 3-oxo-C₁₂:2-HSL

At the bottom, the amount of TNF∝ secreted by Raw264.7 murine cells stimulated by LPS and interferonγ in the presence of PCA,

FIGS. 17A-17C represent in diagram form the gene expression results obtained by measuring the messenger RNA by quantitative PCR for 3 cytokines of interest: Rantes, TNF alpha, IL1-beta,

FIGS. 18A and 18B show in histogram form the cytotoxicity of the AHL 3oxoC₁₂-HSL(A) and 3oxoC₁₂:2-HSL(B) treatments on stimulated Caco-2/TC7 cells. Mean values of different replicates (n≥3)±SEM,

FIGS. 19A-19D show in histogram form the cytotoxicity of the AHL 3-oxo-C₁₂-HSL(A,C) and 3-oxo-C12:2-HSL(B,D) treatments on Raw264.7 murine cells in the basal (A,B) or stimulated (C,D) state. Mean values of different replicates (n=3)±SEM. stimulated,

FIGS. 20A and 20B show in histogram form the LDH secretion under stimulated conditions in the Caco-2/TC7 (left) and Raw264.7 (right) cell lines. Mean values of multiple replicates (n≥6)±SEM,

FIGS. 21A and 21B show in histogram form the LDH secretion under stimulated conditions in the Caco-2/TC7 (A) and Raw264.7 (B) cell lines. Mean values of multiple replicates (n≥6)±SEM,

FIGS. 22A and 22B show in histogram form LDH secretion under stimulated conditions in the Caco-2/TC7 (A) and Raw264.7 (B) cell lines. Mean values of multiple replicates (n≥8)±SEM,

FIGS. 23A and 23B show in histogram form the comparative survival of the E. coli K12 strain after 18 h of incubation in the presence of control molecules or increasing doses of the 3oxoC12-HSL and 3oxoC₁₂:2-HSL AHL. Mean values of different replicates (n=6)±SEM, and

FIG. 24 represents in histogram form the comparative survival of the E. coli K12 strain after 18 h of incubation in the presence of control molecules or 100 μM of molecule. Mean values of different replicates (n=6)±SEM.

The invention is based on the discovery of synthetic bio-inspired analogues of N-acyl-homoserine lactones (AHLs) with an anti-inflammatory activity and an action on the tight junctions at least equal to those of natural AHLs produced in the intestine while having a better stability, a delayed bacterial recognition and a lower toxicity.

The natural AHLs, produced in the intestine, of which the compounds used in the invention are analogs, are the 3-oxo-C₁₂-HSL and the -oxo-C₁₂:2-HSL.

The inventors have segmented these natural AHLs into three areas of interest:
Lactone homoserine head
Oxo substitution to form a ketone at the 3^{rd} carbon of the carbon chain
Acyl chain of 10 to 16 carbons.

In addition, the L-enantiomer of the AHL is the active form (no activity of the D-enantiomer).

The inventors then studied the influence of a modification with various chemical groups of each of these three areas of interest on the biological activity on human and murine cells, on the stability and the bacterial recognition of the analogues thus obtained.

They then discovered that the length of the carbon chain can be 10 and up to 16 carbon atoms, with an optimal length of 14 carbon atoms, and only tolerates the addition of chemical groups of low steric hindrance. The presence of the lactone group at the C3 position is necessary, because its removal or its transformation into an acetal inhibits the anti-inflammatory activity of the AHLs. The head group can only tolerate minor modifications that do not excessively increase its size and retain chemical groups capable of providing hydrogen bonds.

Thus, the compounds used in the invention for the treatment of inflammatory diseases of the epithelium, and in particular inflammatory diseases of the intestine and the psoriasis, have the following general formula I:

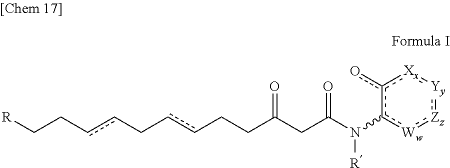

wherein:
X, Y, Z and W are independently of each other a carbon atom or a heteroatom selected from S, N and O, provided that X is different from O,
X, Y, Z and W are independently of each other optionally substituted with a halogen selected from Cl, F, Br, and I, or a linear or branched C₁ to C₄ alkyl group,
x, y, z, and w, independently of each other, are 0 or 1, provided that 3≤x+y+z+w≤4,
R represents H or a linear or branched C₁ to C₄ alkyl group, or a hydroxyl group (OH) or an azido group (N₃),
R′ represents H or a linear or branched C₁ to C₄ alkyl group
[symbol] represents a single or double bond (-cis or trans).

These compounds of formula I can be used in combination with a pharmaceutically acceptable excipient to form a pharmaceutical composition.

The pharmaceutical composition can be formulated for any form of administration, in a solid or liquid or semi-solid form, such as a gel, cream, balm and can be administered orally, rectally, intravenously, or topically.

This pharmaceutical composition is in particular intended for the treatment of an inflammatory disease of the epithelium, and in particular of an inflammatory disease of the intestine and the psoriasis.

The preferred compounds for use in the treatment of an inflammatory disease of the epithelium are the following compounds.

The first of these compounds is the (D/L)-3oxoC12 aminothiolactone (D/L)-3oxoC₁₂-HTL) of the following formula I-1:

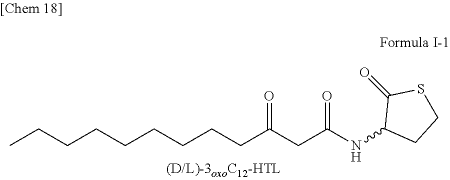 [SMILES: CCCCCCCCCC(=O)CC(=O)NC1CCSC1=O]

The compound of formula I-1 corresponds to the natural 3-oxo-C₁₂ HSL in which the lactone head has been replaced by a thiolactone group.

As shown in FIG. 1, which represents in bar graph form the results of stimulation of Caco-2/TC7 cells by the IL-1β in the presence of the (D/L)-3oxo C₁₂ aminothiolactone of formula I-1 used in the invention, in comparison to DMSO, this compound shows in the human Caco-2/TC7 enterocyte line (cells of the intestinal epithelium) an activity equivalent to that of the natural 3-oxo-C₁₂ HSL, and is more active than the latter at the 100 μM dose.

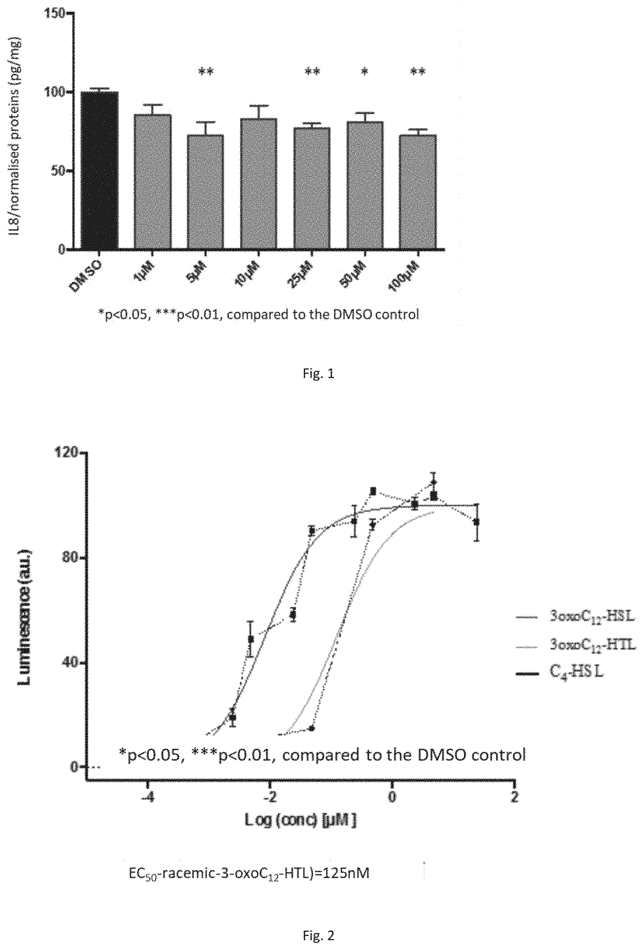

FIG. 2 shows the activation curves of the LasR receptor on bacterial reporter strain in relation to the natural molecules C4-HSL and 3-oxo-C₁₂-HSL and to the molecule of formula I-1 used in the invention.

It can be seen from this FIG. 2 that the racemate of formula I-1 shows a delayed recognition capacity with an EC₅₀ of 125 nM vs. 0.9 nM for the natural molecule 3-oxo-C₁₂-HSL and vs. no recognition (EC₅₀>1000 μM) for the natural molecule C₄-HSL.

The second of these compounds is the (S,S) 3-oxo C₁₂ aminocyclohexanol ((S,S) 3 oxo C₁₂-ACH) of formula I-2 below:

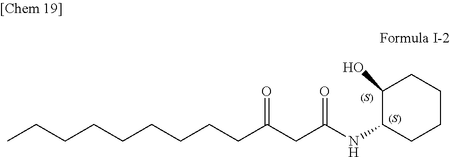

The compound of formula I-2 corresponds to the 3-oxo-C₁₂ HSL in which the lactone head has been replaced by a (S,S)-aminocyclohexanol group.

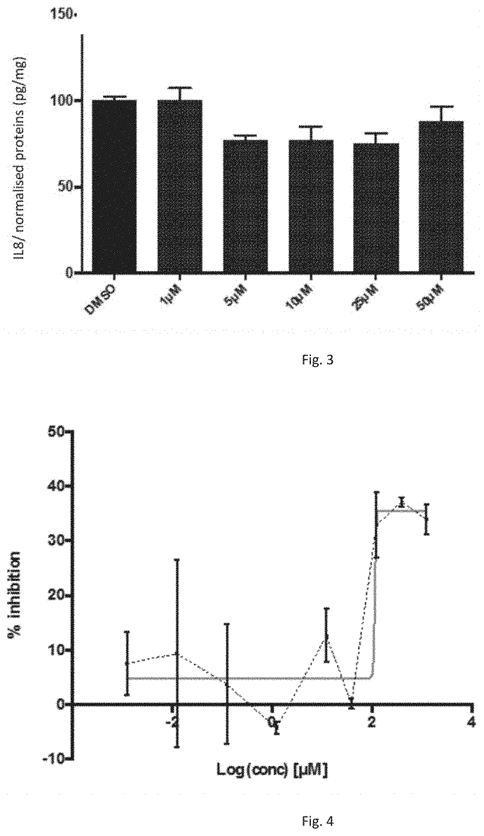

It can be seen from FIG. 3, which represents in bar graph form the results of stimulation of Caco-2/TC7 cells by the IL-1B in presence of the (S,S)-3-oxo C₁₂ aminocyclohexanol ((S,S)-3-oxo C₁₂-ACH) of formula I-2 used in the invention, that this compound exhibits in the human Caco-2/TC7 enterocyte line an activity equivalent to that of the natural 3-oxo-C₁₂ HSL in the 1-50 μM range. As seen in FIG. 4, which represents the inhibition curve of the IL-8 secretion by human keratinocytes stimulated by IL-17 and TNF-α in the presence of increasing doses of the (S,S) 3-oxo C₁₂ aminocyclohexanol ((S,S) 3-oxo C₁₂-ACH) of formula I-2 used in the invention, this compound presents a relative EC₅₀ of 111 nM and a maximum inhibition corresponding to 39% of the inhibition obtained in the presence of the reference compound betamethasone, which allows to show the generalization of the anti-inflammatory effects of this molecule to several cell lines, resulting from various organs of the human body.

It can be seen from FIG. 5, which represents the inhibition curve of the IL-2 secretion by the human lymphocytes stimulated by CD2, CD3 and CD28, in the presence of increasing doses of the (S,S)-3-oxo C₁₂-aminocyclohexanol ((S,S)-3-oxo C₁₂-ACH) of formula I-2 used in the invention, that this compound presents a relative EC₅₀ of 89.9 nM and a maximum inhibition corresponding to 46% of the inhibition obtained in the presence of the reference compound betamethasone, which confirms the generalization of the anti-inflammatory effects of this molecule to several cell lines, resulting from different organs of the human body.

It can also be seen from FIGS. 4 and 5 that the natural AHL of Formulae A and B are inactive in the scope of the tests, the results of which are shown in FIGS. 4 and 5, which demonstrates the superiority of the molecules of Formula I used in the invention.

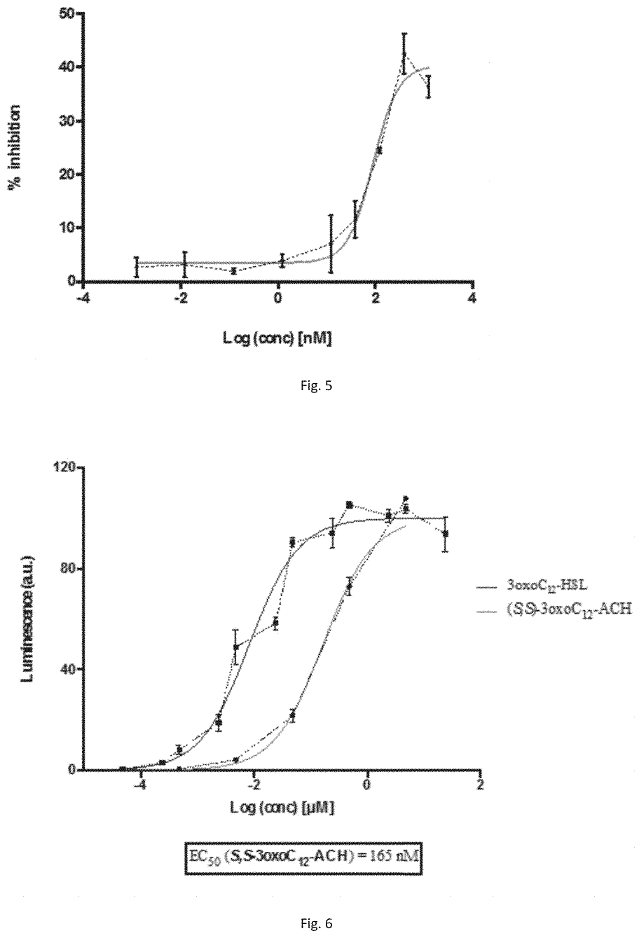

From FIG. 6, which shows the activation curves of the LasR receptor on bacterial reporter strain in relation to the natural 3-oxo-C₁₂HSL molecule and the molecule of formula I-2 used in the invention, the compound of formula I-2 has an EC₅₀ of 165 nM. It therefore has an even more delayed recognition ability than the compound of formula I-1.

Finally, this molecule of formula I-2 is also resistant to the hydrolysis giving an open form (both enzymatic and spontaneous).

FIG. 8 shows in bar graph form the results of stimulation of Raw 264.7 murine cells by the IFN-γ/LPS combination in the presence of (S,S)-3-oxo C₁₂ aminothiolactone ((S,S)-3 oxo C₁₂-ACH) of formula I-2. This figure shows that the compound of formula I-2 is more active than the reference molecule 3-oxo-C₁₂-HSL, at all doses in the concentration range of 1 to 50 μM.

The third compound is (S)-3-oxo C₁₂ aminothiolactone ((S)-3oxoC₁₂-HTL) of the following formula I-3:

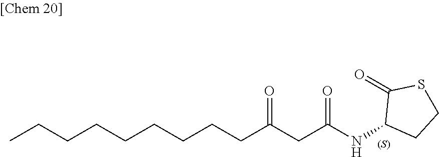

The compound of formula I-3 corresponds to the 3-oxo-C₁₂ HSL in which the lactone head has been replaced by a thiolactone group.

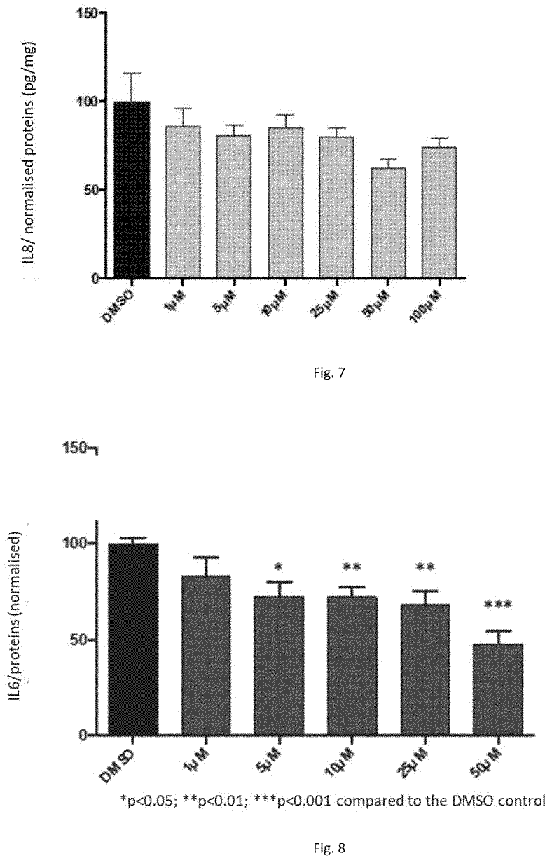

As shown in FIG. 7, which represents in bar graph form the results of stimulation of Caco-2/TC7 cells by the IL-1β in the presence of (S)-3-oxo C₁₂ aminothiolactone ((S)-3oxoC₁₂-HTL) of formula I-3, the effects observed with the molecule of formula I-3 used in the invention are similar to those observed with the natural 3-oxo-C₁₂-HSL molecule, in particular a 27% decrease in the inflammatory secretion at 5 μM.

The fourth compound is (R,S)-3-oxo C12-aminocyclohexanol of the following formula I-4:

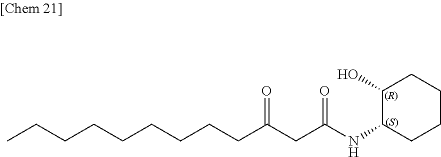

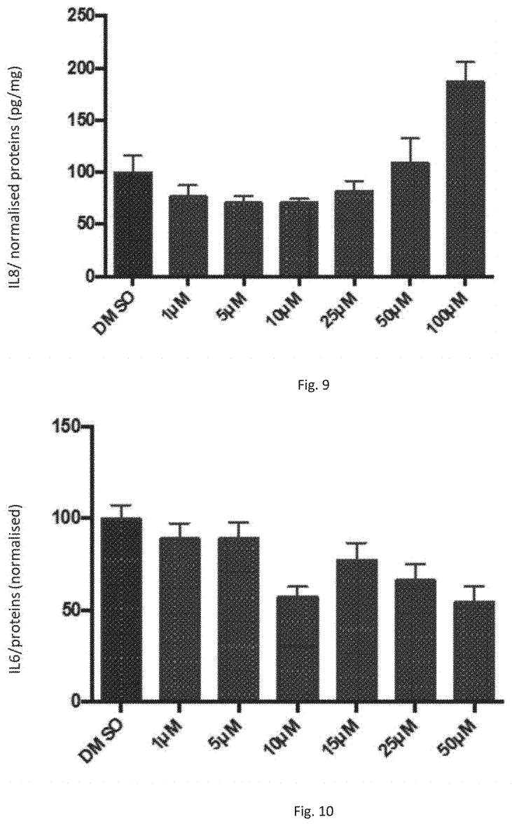

FIG. 9 shows in bar graph form the results of stimulation of Caco-2/TC7 cells by the IL-1β in the presence of the (R,S)-3-oxo C₁₂ aminocyclohexanol ((R,S)-3-oxo C₁₂-ACH) of formula I-4 used in the invention.

From FIG. 9, we see in the human enterocyte line Caco-2/TC7 an activity equivalent to that of the natural 3-oxo-C₁₂ HSL in the 1-50 μM range.

FIG. 10 represents in bar graph form the results of stimulation of Raw 264.7 murine cells by the IFN-γ/LPS combination in the presence of the (R,S) 3-oxo C₁₂-ACH of formula I-4 used in the invention.

It can be seen from FIG. 10 that in the Raw 264.7 murine macrophage line the molecule shows a higher activity than the natural 3-oxo-C₁₂ HSL in the 1-50 μM range.

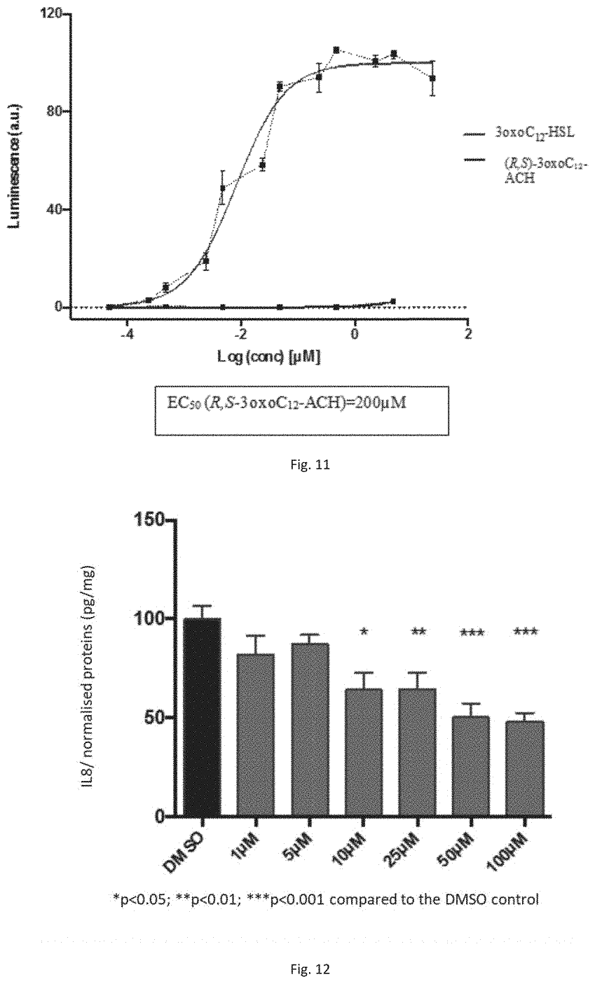

FIG. 11 shows the activation curves of the LasR receptor on bacterial reporter strain in relation to the natural molecule 3-oxo-C₁₂-HSL and to the (R,S) 3-oxo C₁₂-aminocyclohexanol of formula I-4 used in the invention. As can be seen from FIG. 11, the (R,S)-3-oxo C₁₂-aminocyclohexanol of formula I-4 has an EC₅₀ of 200 μM. It therefore has a much-delayed recognition capacity compared to the natural AHLs.
The fifth compound is the 3-oxo C₁₂-aminocyclohexanol of the following formula I-5:

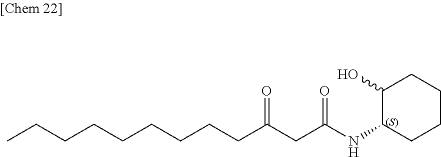

The compound of formula I-5 corresponds to the 3-oxo-C₁₂ HSL in which the lactone head has been replaced by an aminocyclohexanol group.
The sixth compound is the 3-oxo C₁₂-aminochlorophenol of the following formula I-6:

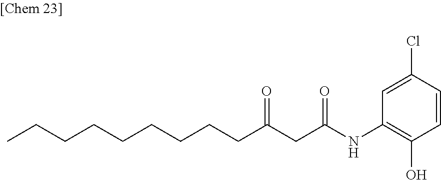 [SMILES: CCCCCCCCCC(=O)CC(=O)Nc1cc(Cl)ccc1O]

The compound of formula I-6 corresponds to the 3-oxo-C₁₂ HSL in which the lactone head has been replaced by an aminochlorophenol group.

FIG. 12 shows in bar graph form the results of stimulation of Caco-2/TC7 cells by the IL-1β in the presence of the 3-oxo C₁₂-aminochlorophenol of formula I-6 used in the invention.

It can be seen from FIG. 12 that in the human enterocyte line Caco-2/TC7 the molecule shows a significantly higher activity than that of the natural 3-oxo-C₁₂ HSL in the 10-100 μM range.

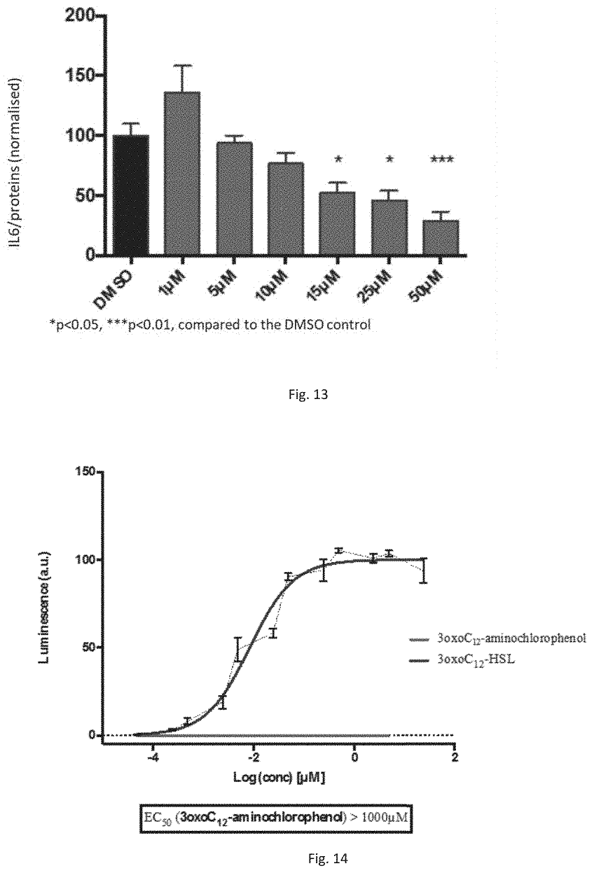

FIG. 13 represents in bar graph form the results of stimulation of Raw 264.7 murine cells by the IFN-γ/LPS combination in the presence of the 3-oxo C₁₂-aminochlorophenol of formula I-6 used in the invention.

As can be seen, the molecule of formula I-6 shows in the Raw 264.7 murine macrophage line a significantly higher activity than that of the natural 3-oxo-C₁₂ HSL in the 10-50 μM range.

FIG. 14 shows the activation curves of the LasR receptor on bacterial reporter strain in relation to the natural molecule 3-oxo-C₁₂-HSL and to the 3-oxo C₁₂-aminochlorophenol of formula I-6 used in the invention.

As can be seen, the molecule of formula I-6 has an EC₅₀ greater than 1000 μM. It has the most delayed recognition ability compared to the natural AHLs and to the molecules of formula I-1 to 1-5 used in the invention.

### EXAMPLES

1. Experimental Procedures and Protocols Used in Biology
The penicillin-streptomycin antibiotics, the nonessential amino acids (NEAA), and the L-glutamine were from Invitrogen (Thermo Fisher Scientific, Waltham, USA). The saline solution buffered with Dulbecco's phosphate (DPBS 10×), the high glucose cell culture medium (DMEM GlutaMAX 4.5 g/L glucose), the DMEM and the DMEM without phenol red were from Gibco (Thermo Fisher Scientific, Waltham, Mass., USA). The foetal calf serum was from GE Healthcare (Life Sciences, South Logan, Utah, USA).
The 2-Hydroxyquinoline (CAS [59-31-4]), the 3oxo C₁₂-HSL molecule, and the sterile DMSO were purchased from Sigma. The molecule 3oxoC₁₂: 2-HSL was synthesized on demand by Diverchim (Roissy-en-France, France)
All the absorbance and luminescence tests were read on the SpectraMax M5 spectrometers from Molecular Devices®.

1.1. Cell Culture

1.1.1. The Caco-2/TC7 Cell Line
The Caco-2 cell line is derived from a human colon adenocarcinoma and represents a cell culture model of enterocytes lining the epithelium of the small intestinal. The Caco-2/TC7 cell line is a clonal population of Caco-2 cells that reproduces to a large extent and homogeneously most of the morphological and functional characteristics of the normal human enterocytes.
The Caco-2/TC7 cells exhibit a contact inhibition property leading to a growth arrest when the cells reach the confluence, which allows the establishment of a cell monolayer. During the exponential growth phase (from seeding to confluence), the cells remain undifferentiated. At confluence, they can spontaneously (in the absence of differentiation inducers) differentiate and progressively polarize. The differentiation process, which is a growth-related mechanism, is maximal at the end of confluence (stationary phase of the growth curve).
The cells develop a brush border at the apical pole containing microvilli and with characteristic enterocytic enzymes, such as hydrolases.
In accordance with the published literature, the Caco-2/TC7 cells in our experiments were seeded at 10⁵ cells/well (equivalent to 10-12×10³ cells/cm²) in 6-well plastic culture plates. The cells were maintained in a glucose-rich medium (high glucose in the DMEM GlutaMAX medium 4.5 g/I glucose) supplemented with 20% heat-inactivated foetal calf serum, 1% nonessential amino acids NEAA, and 1% penicillin streptomycin. The cells were grown at 37° C. in a 10% CO₂/air atmosphere. The medium was changed every day. Under these conditions, the cells were confluent on day 6. On day 17, the cells were serum-starved, and were used on day 18.

1.1.2. The Raw 264.7 Cell Line
The Raw 264.7 cell line consists of murine cells of macrophage type derived from cell line transformed by the virus of the Abelson leukaemia and providing from BALB/c mice. This cell line is very often used as a model of macrophages in vitro. The Raw 264.7 cells are capable of a phagocytosis and a pinocytosis, can kill the target cells by antibody-dependent cytotoxicity, and secrete a wide range of inflammatory cytokines as well as nitric oxide (NO). In addition, the Raw 264.7 cells are a very convenient cell line: the cells grow rapidly, appreciate the small diameter wells, and should be used before confluence. These conditions make it an advantageous line for the compound screening.

The cells were used between the passages 13 and 26.
The Raw 264.7 cells used are from the ATCC bank. They were grown in DMEM supplemented with 10% heat-inactivated foetal calf serum and 1% L-glutamine to 200 mM, and maintained at 37° C. with a 5% CO2/air atmosphere. The medium was changed every two days.

1.1.3. The Bacterial Reporter Strain E. coli pSB1075
A bacterial reporter strain of the Quorum Sensing was used to study the ability of the molecules to be recognized by the Pseudomonas aeruginosa AHL receptor (LasR) and induce an activation. Thus, the strain pSB1075 of Escherichia coli was used.
The enterobacterium Escherichia coli does not naturally produce AHL, nor does it have an orphan receptor capable of recognizing AHL. This bioluminescent strain was modified by addition of a plasmid pSB1075. This plasmid contains genes encoding both tetracycline resistance and the expression of the LasR AHL receptor from the Pseudomonas aeruginosa bacteria, as well as a fusion gene derived from the LasR promoter and the luxCDABE gene from Photorhabdus luminescence. The bacterial strain was grown in the LB medium supplemented with 5 μg/ml tetracycline to exert a selection pressure and ensure that only the desired strain was amplified. The fusion gene included in the plasmid gives the bacteria the ability to emit a bioluminescence when the LasR receptor is activated, and provides the user with a robust test for the molecular screening. The LasR receptor of P. aeruginosa is well suited for the recognition of the long-chain AHL, particularly its natural partner, the 3oxoC₁₂-HSL, which causes the highest bioluminescent response. In contrast, short-chain AHL such as the C₄-HSL do not induce the bioluminescence emission.
Briefly, the bacterial culture was started on day 0 in 10 ml LB medium supplemented with 5 μg/ml tetracycline and maintained for 24 hours at 37° C. and under stirring at 70 rpm. On day 1, the culture was diluted 1:100 (P1) in 10 ml of LB medium supplemented with 5 μg/ml tetracycline and maintained for 24 hours at 37° C. under stirring at 70 rpm. On day 2, the experiment took place: a bacterial suspension extemporaneously diluted 1:10 in LB medium supplemented with 5 μg/ml tetracycline (P2) was dispensed into a black opaque 96-well plate and incubated for 4 hours with a range of AHL concentrations or controls until the resulting luminescence was read at the end point. All experiments were performed in triplicates.

1.1.4. Bactericidal Dosage
The K12 strain of E. coli was grown on agar on day 0 and a colony was transferred to the LYBHI liquid bacterial culture medium on day 1. On day 2, the colony was diluted 1:100 in LYBHI medium and amplified for 18 hours before transfer to an opaque 96-well plate. In each well, LYBHI, controls or increasing doses of tested molecules and bacteria were distributed. The absorbance at 600 nm was read at start-up (t=0) and after an 18-hour incubation. The raw absorbance values were corrected using the absorbance of solutions without bacteria. All experiments were performed twice, each with 4 replicates.

1.2. Evaluation of the Biological Activity of the Molecules in the Mammalian Cells

1.2.1. Stimulation of Caco-2/TC7 with Cytokines
The Caco-2/TC7 cells were seeded in 6-well plates at 100,000 cells/well and grown for 18 days. On day 17, the cells were serum-starved, which means that the cell medium was replaced with a foetal calf serum-free medium, and used on day 18.
The stimulation medium was composed of a medium referred to as “starvation medium”, i.e. without foetal calf serum (DMEM GlutaMAX, 1% NEAA, 1% penicillin-streptomycin) with 100 μM of 2-HQ. The cells were incubated for 18 h at 37° C. with 2 ml of stimulation medium containing 0.1% DMSO (negative control) or stimulation medium containing the compounds tested at desired concentrations, with or without proinflammatory cytokines to induce an inflammation. To induce the inflammation, either the IL-1β at 25 ng/ml or the combination of TNF-α and IFN-γ at 50 ng/ml each were used. After 18 h, the supernatants were collected and stored at −80° C. before analysis by ELISA test. The cells were washed with 1 ml of PBS 1×/well, and lysed in 100 μl of PBS 1× containing 1% Triton X-100. The cells were harvested by scraping and stored at −80° C. before the quantification of the protein. The dosing of the LDH was performed immediately before freezing.
All the experiments on the cells were performed in triplicates.

1.2.2. Stimulation of Raw 264.7 with LIPS and IFN-γ
Raw 264.7 cells were seeded in 12-well plates at 75,000 cells/well, or 24-well plates at 40,000 cells/well, to reach 80-90% confluence after 3 days of culture. For the stimulation, the cells were incubated for 6 h at 37° C. with 750 μL (respectively 500 μL) of 100 μM 2-HQ-enriched cell medium containing 0.1% DMSO (negative control) or compounds tested at desired concentrations, with or without LPS (10 ng/ml) and IFN-γ (20 U/ml) to establish an inflammation. After 6 h, the supernatants were collected and stored at −80° C. before analysis by ELISA test. The cells were harvested by scraping in 100 μL of PBS 1×/well and stored at −80° C. before the quantification of the total protein. The dosing of the LDH was performed immediately before freezing.
All experiments were performed in triplicates.

1.2.3. Measurement of Protein Concentration in Cell Lysate
The total protein concentrations were determined in cell lysates using the assay reagents of the protein which are bicinchoninic acid (BCA) and bovine serum albumin (BSA) according to the instructions of the manufacturer (Uptima-Interchim, Montlugon, France).

1.2.4. Quantification of the Human Cytokines by ELISA
The levels of the proinflammatory cytokine IL-8 produced by the cells were determined in the cell supernatants and/or cell lysates using the commercially available IL-8 ELISA detection kit (Duoset Human C×CL8/IL-8, ref. DY208) provided by R & D Systems (Minneapolis, Minn., USA) according to the instructions of the manufacturer.
All the cytokine levels were first normalized to the protein content determined in the corresponding cell lysates. Then, to compare the experiments, they could be further normalized using the activated control condition (DMSO+cytokines) as the 100% response.

1.2.5. Quantification of the Murine Cytokines
The levels of IL-6 murine cytokines produced by cells were determined in cell supernatants or lysates using the commercially available “BD OptEIA Mouse IL-6 ELISA Set” from BD Biosciences (San Jose, Calif., USA, ref. 555240). All the ELISA kits were used according to the instructions of the manufacturer.
The cytokine rates were first normalized to the protein content determined in the corresponding cell lysates. To compare the experiments, they could be further normalized by using the activated control condition (DMSO+cytokines) as the 100% response.

1.2.6. Cytotoxicity Test
The cytotoxicity of the tested compounds and of the controls, with or without proinflammatory cytokines, was assessed using a release test of the lactate dehydrogenase (LDH), which tracks the release of this enzyme in the cell supernatants, a good indicator of the damages of the membrane and of the cell death. A compound was considered cytotoxic when its secreted LDH rate was greater than 10%.
Two methods can be used to perform the test: measurement with a pyruvate/NADH solution (Sigma), or using the Cytotoxicity detection kit^{PLUS} (LDH) of Roche (Sigma-Aldrich).
For the Pyruvate/NADH method, a pyruvate/NADH solution was prepared with 4.1 mg pyruvic acid (0.62 mM) and 7.7 mg NADH (0.18 mM) in 60 ml of 0.1 M PBS (pH 7.4).
To measure the concentration of LDH in the supernatant, 800 μL of NADH was added to 200 μL of supernatant in a plastic cuvette and the decrease in the absorbance at 340 nm was monitored for 1 min. To measure the concentration of LDH in the cell lysate, 800 μL of NADH was added to 10 μL of supernatant and 190 μL of 0.1 M PBS in a plastic cuvette, and the decrease in the absorbance at 340 nm was monitored for 1 min. The percentage of LDH released in the supernatant was calculated as the ratio of the corrected slopes in the supernatant and the cell lysate.
Using the Cytotoxicity Detection Kit^{PLUS} (LDH-Roche-Ref. 04744934001), the LDH levels were determined in the cell supernatants and the lysates by means of the absorption-based and colorimetric test, and performed according to the instructions of the manufacturer. The percentage of cytotoxicity could be established with the formula:

$${\%}_{LDH} = \frac{{DO}_{sample} - {DO}_{low control}}{{DO}_{high control} - {DO}_{low control}} \times 100 \qquad [ Math 1 ]$$

1.3. Biological Activity of the Molecules on the Bacterial Reporter Strain E. coli pSB1075
Escherichia coli does not naturally produce AHL. This bioluminescent strain was developed by the addition of a plasmid pSB1075 containing genes encoding the tetracycline resistance and the expression of the LasR AHL receptor, as well as a fusion gene of the LasR promoter and the luxCDABE gene of Photorhabdus luminescence.
On day 1, a 1/100 dilution of the bacterial strain (P1) was grown for 24 hours at 37° C. under stirring (70 rpm) in a LB medium containing 5 μg/ml tetracycline (pressure selective). On day 2, P1 was diluted 1:10 in the same medium to obtain P2. In a black 96-well plate were placed 200 μL of P2 and 10 μL of the short chain compound (medium, water and DMSO for the negative controls, C4-HSL as positive control, and the test sample at the desired concentration). The plate was incubated for 4 hours at 37° C. under stirring at 70 rpm. The luminescence was then read at all wavelengths (integration time 200 ms) on a microplate reader.
For the competition assays the culture protocol was similar but the bacteria were first pre-incubated with 1, 10 or 100 nM of 3oxoC₁₂-HSL for different durations (1, 2, 6 or 16 h) before dilution to obtain P2. After that, the incubation was continued according to the conventional protocol, with the test compound at the desired concentrations.

1.4. Statistical Analysis
All the data are represented as the mean plus or minus SEM of n independent experiments, and were tested for the Gaussian distribution. The statistical significance was examined by a Student test t, a one-way ANOVA, a two-way ANOVA, or a Kruskal-Wallis test, depending on the data set, combined with a post-test (Tukey or Dunn multiple comparison test). The differences were considered significant when p<0.05. All the statistical analyses were performed using the Prism 6.0, GraphPad software.

2. Materials and Methods in Chemistry
Unless otherwise stated, all the reactions were performed under argon atmosphere in dry glassware. The reagents were purchased from commercial suppliers Sigma-Aldrich and TCI Chemicals and were used without further purification.
The flash chromatography was performed on pre-packaged silica gel columns (40-63 μm irregular SiO₂ silica gel) of CHROMABOND® Flash (Macherey-Nagel, Duren, Germany), mounted on an automated SPOT platform from ArMen.
The thin layer chromatography was performed on aluminium sheets coated with silica gel 60 F₂₅₄ (Millipore, Merck) and revealed with potassium permanganate (KMnO4), iodine on silica, bromocresol green or under an UV light (254 nm or 365 nm).

2.1. Convention for the Numbering of the Atoms in N-Acyl Homoserine Lactones and their Analogues
the numbering of the atoms adopted in the AHL molecules and their analogues is as follows:

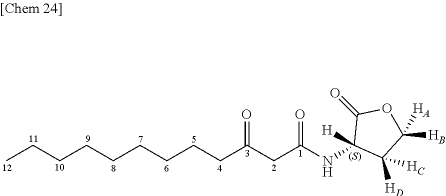

2.2. Experimental Procedures for the Synthesis and Physicochemical Characterization of the Natural AHL, Intermediates and Non-Natural Analogues

General Procedure for the Preparation of Meldrum 3a-b Acid Derivatives (GP1)
The appropriate carboxylic acid (1.0 equiv.) was dissolved in dichloromethane (denoted DCM) (1.5 ml/mmol acid) at room temperature. DCC (1.1 equiv.), DMAP (1.05 eq.), and Meldrum acid (1.0 eq.) were added to the mixture sequentially. The reaction mixture was stirred overnight at room temperature under argon atmosphere. The reaction was monitored by TLC in a 1:1 EtOAc/cyclohexane mixture and revealed with iodine.
After completion of the reaction, the reaction mixture was filtered to remove the precipitated DCU and the filtration residue thoroughly washed with dichloromethane. The filtrate was collected and the solvent removed under vacuum. The resulting oil was diluted in EtOAc and the organic phase was extracted with HCl 1 M (×2), while the aqueous phase was washed with EtOAc (×2). The combined organic phases are dried over MgSO₄, before removing the solvent under vacuum. The raw product, obtained in the form of oil, was directly engaged in the next step.

### 2,2-dimethyl-5-(1-oxodecyl)-1,3-dioxane-4,6-dione 3a [182359-65-5]

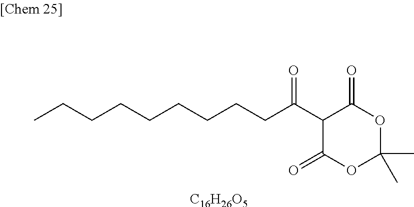 [SMILES: CCCCCCCCCC(=O)C1C(=O)OC(C)(C)OC1=O ; "C16H26O5"] [0214] Prepared by GP1 with a yield of 94%. ¹H RMN (300 MHz, CDCl₃): δ:12-3.03 (m, 2H, C (4)H₂), 1.74 (s, 6H, OC(CH₃)₂O), 1.45-1:24 (m, 14H C (5) H₂ to C (11) H₂), 0.91 to 0.86 (m, 3H, C (12) H₃). ¹³C RMN (75 MHz, CDCl₃): δ 198.32 (C(3)), 170.57 (ester), 160.18 (ester), 104.74 (OC(CH₃)₂O), 91.23, 35.74, 31.83, 29.37, 26.76, 26.15, 22.65, 14.08 (C (12)). R_{f} (1:3 EtOAc/Cychex): 0.22.

### 2,2-dimethyl-5-(1-oxodecyl)-1,3-dioxane-4,6-dione 3b azide

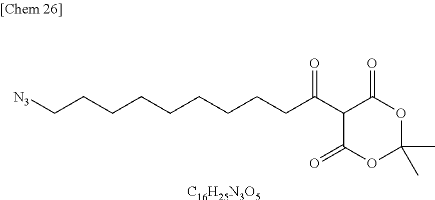 [SMILES: CC1(C)OC(=O)C(C(=O)CCCCCCCCCN=[N+]=[N-])C(=O)O1 ; "C16H25N3O5"]

Prepared by GP1 with a yield of 87%. ¹H RMN (300 MHz, chloroform-d): 3.24 (t, J=6.9 Hz, 2H, C (12)H), 3:08-3:02 (m, 2H, C (4) H₂), 2.12 (s, 1H, C (2) H), 1.72 (s, 6H, C(CH₃)₂), 1.62-1.54 (m, 4H, C (5) H₂ and C(6)H₂, 1.31-1.28 (m, 10H, C (7) H₂ to C (11) H₂). ¹³C RMN (75 MHz, CDCl₃): δ198.33 (ketone), 170.68 (ester), 160.30 (ester), 104.88 (C₂), 91.36 (OC(CH3)₂O), 51.56 (C₁₂, 43.87 (C₄), 35.82 (C₅), 29.37, 29.17, 28.92, 26.91 ((CH₃)₂), 26.77, 26.20, 23.91. R_{f} (1:3 EtOAc/Cychex): 0,25.

General Procedure for the Methanolysis of the Meldrum 4a-b Acid Derivative (GP2)
The meldrum 3a-c acid derivative (1.0 equiv.) was dissolved in excess methanol and the reaction flask was equipped with a reflux apparatus under an argon atmosphere. The reaction was heated at reflux for 2 hours, then the heating was stopped and the mixture allowed to cool spontaneously to room temperature and stirred overnight. The progress of the reaction was followed by TLC in an EtOAc/cyclohexane mixture at 1:1 and revealed in iodine. On completion, the solvent was removed under vacuum and the oily product was used raw in the following step.

### 3-oxododecanoate of methyl 4a [76835-64-8]

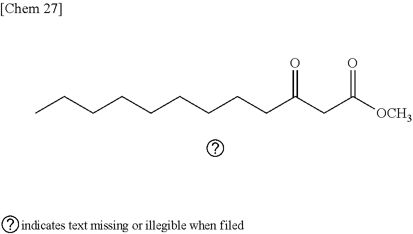 [SMILES: CCCCCCCCCC(=O)CC(=O)OC] [0220] Prepared by GP2 with a yield of 96%. ¹H RMN (300 MHz, CDCl₃) 3.75 (s, 3H, OCH₃), 3.46 (s, 2H, C (2) H₂), 2.54 (t, J=7.4 Hz, 2H, C (4) H₂), 1.59 (d, J=7.4 Hz, 2H, C (5) H₂), 1.28 (s, 12H, C (6) H₂ to C (11) H₂), 0.92 to 0.87 (m, 3H, C (12) H₃). ¹³C RMN (75 MHz, CDCl₃): δ202.86 (C₃), 167.70 (C₁), 52.31 (OCH₃), 49.01, 43.09, 31.85, 29.39, 29.34, 29.24, 29.00, 23.47, 22.66, 14:09 (C₁₂). R_{f} (1:3 EtOAc/Cychex): 0.55.

### Methyl ester of the acid 12-Azido-3-oxododecanoic 4b [1421598-01-7]

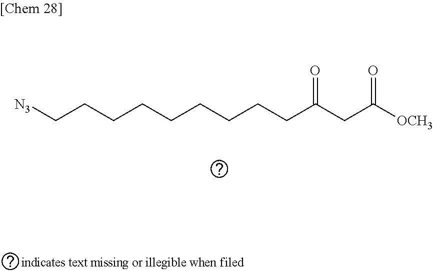 [SMILES: COC(=O)CC(=O)CCCCCCCCCN=[N+]=[N-]] [0222] Prepared by GP2 with a yield of 81%. ¹H RMN (300 MHz, CDCl₃): δ3.71 (s, 3H, OCH₃), 3.42 (s, 2H, C (2) H₂), 3.22 (t, J=6.9 Hz, 2H, C (12) H₂), 2.50 (t, J=7.3 Hz, 2H, C (4) H₂), 01.55 (q, J=6.8 Hz, 2H, C (5) H₂), 1.27 (t, J=4.7 Hz, 12H, C (6) H₂ to C (11) H₂). ¹³C RMN (75 MHz, CDCl₃): 202.85, 167.77, 52.40, 51.56, 49.11, 43.12, 29.36, 29.30, 29.14, 29.02, 28.91, 26.77, 23.50. R_{f} (1:3 EtOAc/cyclohexane): 0.6.

General Procedure for Installing an Acetal in Position C₃ 5a-b (GP3)
The ester 3-ketomethyl 4a-b (1.0 equiv.) was diluted in toluene (about 1 ml/mmol ester) and camphorsulfonic acid (0.2 eq.), trimethylorthoformate (5.0 eq.), and ethylene glycol (8.9 eq.) were successively added. The reaction mixture was heated to 80° C. for 3 hours, allowed to cool spontaneously and stirred overnight at room temperature. The reaction was followed by TLC in an EtOAc/cyclohexane 1:6 mixture and revealed with iodine. On completion, the toluene was removed under vacuum and the resulting oil was dissolved in DCM. The organic phase was extracted with a saturated Na—HCO3 solution (×3) while the aqueous phase was washed with DCM. The combined organic phases were dried over MgSO₄ and the solvent removed under vacuum.
If necessary, the oily product was purified by Flash Chromatography on a silica column using an elution gradient of 1:8 to 1:2 EtOAc/cyclohexane.

### Methyl ester of the acid 2-Nonyl-1,3-dioxolane-2-acetic 5a [109873-29-2]

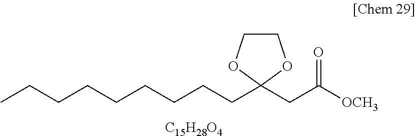

Prepared by GP3 with a yield of 95%. ¹H RMN (300 MHz, CDCl₃): δ04:01-3.95 (m, 4H, OCH₂CH₂O), 3.70 (s, 3H, OCH₃), 2.67 (s, 2H, C (2) H₂), 1.83-1.76 (m, 2H, C (4) H₂), 1.41-1:36 (m, 2H, C (5) H₂), 1.28 (d, J=5.4 Hz, 12H, C (6) H₂ to C (11) H₂), 0.91-0.85 (m, 3H, C (12) H₃). ¹³C RMN (75 MHz, CDCl₃): δ170.05 (C₁), 109.41 (C₃), 65.10 (ketal), 51.72 (OCH₃), 42.42, 37.75, 31.88, 29.68, 29.56, 29.51, 29.29, 23.51, 22.67, 14.10 (C₁₂). R_{f} (1:8 EtOAc/Cychex): 0.31.

### Methyl ester of the acid 2-(9-azido) nonyl-1,3-dioxolane-2-acetic 5b

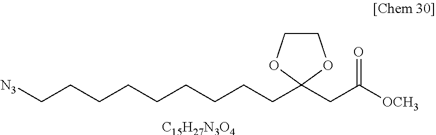 [0229] Prepared by GP3 with a yield of 93%. ¹H RMN (300 MHz, CDCl₃): δ04:00-3.93 (m, 4H, OCH₂CH₂O), 3.67 (d, J=0.9 Hz, 3H, OCH₃), 3.23 (t, J=6.9 Hz, 2H, C (12) H₂), 2.64 (s, 2H, C (2) H₂), 1.80-1.74 (m, 2H, C (4) H₂), 1.61-1.55 (m, 2H C (5) H₂), 1.28 (d, J=5.3 Hz, 12H, C(6) H₂ to C(11) H₂). ¹³C RMN (75 MHz, CDCl₃): δ170.15 (C₁), 109.49 (C₃), 65.22 (acetal), 51.87 (OCH₃), 51.59 (C₁₂), 42.52, 37.81, 29.71, 29.51, 29.45, 29.20, 28.93, 26.79, 23.56. R_{f} (1:4 EtOAc/Cychex): 0.45.

General Procedure for the Basic Hydrolysis of the Methyl Ester 6a-b (GP4)
The ketal-protected methyl ester 5a-b (1.0 eq.) was dissolved in THF (1.25 ml/mmol ester) and a NaOH 1 M solution (about 2.5 eq.) was added. The reaction mixture was refluxed for 3 hours.
The progress of the reaction was followed by TLC in EtOAc/cyclohexane at 1:8 and revealed with iodine.
Once completed and after the reaction mixture was cooled to room temperature, the pH was fixed at 4-5 with HCl IM. The organic phase was extracted with DCM (×3). The combined organic phases were dried over MgSO₄ and the solvent removed under vacuum to give the desired product as an oil.

### Acid 2-Nonyl-1,3-dioxolane-2-acetic 6a [596104-60-8]

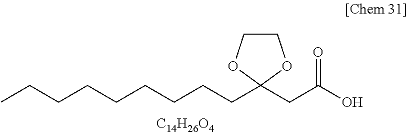 [0235] Prepared by GP4 with a yield of 95%. ¹H RMN (300 MHz, CDCl₃): δ10.93 (s, 1H, OH), 4:06-3.99 (m, 4H, OCH₂CH₂O), 2.72 (s, 2H, C (2) H₂), 1.85-1.78 (m, 2H, C (4) H₂), 1.43-1.37 (m, 2H, C (5) H₂) 1.29 (d, J=5.2 Hz, 12H, C (6) H₂ to C (11) H₂), 0.92-0.87 (m, 3H, C (12) H₃). ¹³C RMN (75 MHz, CDCl₃): δ174.61 (C₁), 109.32 (C₃), 65.10 (OCH₂CH₂O), 42.36 (C₂), 37.61 (C₄), 31.88, 29.65, 29.51, 29.30, 26.91, 23.51, 22.67, 14.10 (C₁₂). Does not migrate under normal TLC conditions.

### Acid 2-(9-azido) nonyl-1,3-dioxolane-2-acetic 6b [1421598-02-8]

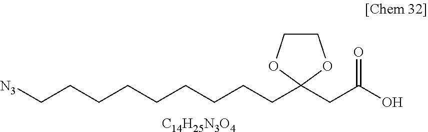 [0237] Prepared by GP4 with a yield of 97%. ¹H RMN (300 MHz, CDCl₃): δ8.77 (s, 1H, OH), 4.07 to 3.93 (m, 4H, OCH₂CH₂O), 3.24 (t, J=6.9 Hz, 2H, C (12) H₂), 2.68 (s, 2H, C (2) H₂), 1.95 to 1.82 (m, 2H, C (4) H₂), 1.61 to 1.55 (m, 2H, C (5) H₂), 1.29 (d, J=6.5 Hz, 12H, C (6) H₂ to C (11) H₂). ¹³C RMN (75 MHz, CDCl₃): δ174.32 (C₁), 109.42 (C₃), 65.21 (acetal), 51.59 (C₁₂), 42.50, 37.66, 29.68, 29.51, 29.45, 29.20, 28.93, 26.19, 23.56. Does not migrate under normal TLC conditions.

General Preparation of Ketoamides Using EDC and DMAP with Various Head Group Facilities (GP5)
For the general head groups, the ketal-protected carboxylic acid 6a (1.0 equiv.) was dissolved in DCM (about 7 ml/mmol acid) and were added sequentially: EDC (1.2 eq.), DMAP (1.7 eq.) and the appropriate amino head group (1.3 eq.).
For the (S)-(−)-(α)-amino-(γ) butyrolactone, the ketal-protected acids 6a-b (1.0 equiv.) and the EDC (1.1 equiv.) were dissolved in DCM (about 2 ml/mmol substrate) under argon atmosphere and left under stirring at room temperature for 20 min. Then hydrochloride (S)-(−)-(α)-amino-(γ) butyrolactone (1.3 eq.) and DMAP (1.7 eq.) in DCM (about 2 ml/mmol substrate) were added. The reaction was stirred for 12-22 hours under argon atmosphere at room temperature.
The progress of the reaction was followed by TLC in an EtOAc/cyclohexane mixture and the revelation was obtained with iodine, an UV light, potassium permanganate or bromocresol green, depending on the nature of the head group. Upon completion of the reaction, an additional DCM was added (13 ml/mmol substrate) and the organic phase was washed with HCl 1 M (×3). The phases were separated, the combined organic phases were dried over MgSO₄ and the solvent removed in vacuo to give the desired compound as an oil. If necessary, the product was purified by flash chromatography on a silica column.

Alternative Procedure Using ECD and Et₃N for the Preparation of Ketoamides from (S)-(−)-(α)-Amino-(γ) Butyrolactone (GP5)

A solution of (S)-(−)-(α)-amino-(γ) hydrochloride butyrolactone (0.91 eq.) in DCM (approx. 6 ml/mmol substrate) was stirred. Et₃N (1.0 eq.), the protected acid 6a (1.0 eq.) and the EDC (1.37 eq.) were added successively. The reaction mixture was stirred at room temperature for 40 hours.
The reaction was followed by TLC in EtOAc/Cychex 1:2 and revealed with iodine, potassium permanganate and UV light. Upon completion, the mixture was evaporated to dryness under vacuum. The residue was partitioned between water (8 ml/mmol substrate) and EtOAc (17 ml/mmol substrate), and the organic phase was washed successively with a saturated solution of NaHCO3 (×2) and brine (×2). The organic layer was dried over MgSO₄ and evaporated to dryness to give the desired compound as an oil. If necessary, the product could be purified by flash chromatography on a silica column.

### (S)-2-(2-nonyl-1,3-dioxolan-2-yl)-N-(2-oxotetrahydrofuran-3-yl) acetamide 7a [182359-61-1]

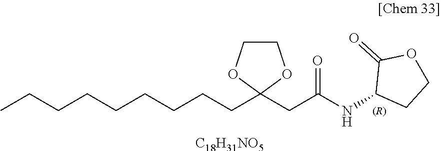

Prepared by GP5 and GP5bis with yields of 88% and 91% respectively
¹H RMN (300 MHz, CDCl₃): δ6.99 (d, J=6.3 Hz, 1H, NH), 04.58 (ddd, J=11.6, 8.6, 6.3 Hz, 1H, C_{α}H), 4.46 (td, J=9.1, 1.3 Hz, 1H, H_{A}), 4.27 (ddd, J=11.2, 9.1, 5.9 Hz, 1H, H_{B}), 4.11 to 3.96 (m, 4H, OCH₂CH₂O), 2.80 (dddd, J=12.5, 8.7, 5.9, 1.3 Hz, 1H, H_{D}), 2.64 (s, 2H, C (2)H₂), 2.13 (dtd, J=12.5, 11.3, 8.8 Hz, 1H, H_{C}), 1.71-1.65 (m, 2H, C (4)H₂), 1.39-1.33 (m, 2H, C (5) H₂), 1.27 (d, J=7.0 Hz, 12H, C (6) H₂ to C (11) H₂), 0.91-0.84 (m, 3H, C (12) H₃). ¹³C RMN (75 MHz, CDCl₃): δ175.35 (C₁), 169.95 (ester), 109.76 (C₃), 66.06 (CH_{A}H_{B}), 65.25 (CH₂ acetal), 65.12 (CH₂ acetal), 49.11 (CH), 44.33, 37.66, 32.02, 30.53 (CH_{C} H_{D}), 29.82, 29.67, 29.65, 29.45, 29.85, 22.82, 14.26 (C₁₂). R_{f} (4:1 EtOAc/Cychex): 0,35. HRMS (ESI): exact mass calculated for C₁₈H₃₁NO₅Na ([M+Na]⁺): 362.2094. Found 364.2095.

### 12-azido-3-(1,3-dioxolane)-N-((3S)-tetrahydro-2-oxo-3-furanyl) dodecanamide 7b

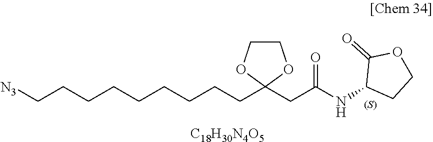 [0249] Prepared by GP5 with a yield of 69%. ¹H RMN (300 MHz, CDCl₃: δ.99 (d, J=6.4 Hz, 1H, NH), 4.57 (ddd, J=11.6, 8.7, 6.4 Hz, 1H, C_{α}H), 4.45 (td, J=9.1, 1.3 Hz, 1H, H_{A}), 4.26 (ddd, J=11.1, 9.1, 5.9 Hz, 1H, H_{B}), 4.08-3.95 (m, 4H, OCH₂CH₂O), 3.24 (t, J=6.9 Hz, 2H, C (12)H₂), 2.84 to 2.73 (m, 1H, H_{D}), 2.63 (s, 2H, C (2)H₂), 2.13 (dtd, J=12.5, 11.4, 8.9 Hz, 1H, H_{C}), 1.71 to 1.65 (m, 2H, C (4)H₂), 1.63 to 1.53 (m, 2H, C (5)H₂), 1:38-1:28 (m, 12H, C (6)H₂ to C (11)H₂). ¹³C RMN (75 MHz, CDCl₃): δ175.33 (C₁), 169.90 (ester), 109.71 (C₃), 66.03 (CH_{A}H_{B}), 65.23 (CH₂ acetal), 65.11 (CH2 acetal), 51.60 (C₁₂), 49.07 (CH), 44.31, 37.60, 30.41 (CH_{C}H_{D}), 29.72, 29.48, 29.45, 29.21, 28.94, 26.80, 23.77. R_{f} (2:1 EtOAc/cyclohexane): 0.20. HRMS (ESI): exact mass calculated for C₁₈H₃₀N₄O₄Na ([M+Na]⁺): 405.2108. Found: 405.2110.

### 2-nonyl-N-(3-tetrahydro-2-oxo-3-thienyl)-1,3-dioxalane-2-acetamide 7f

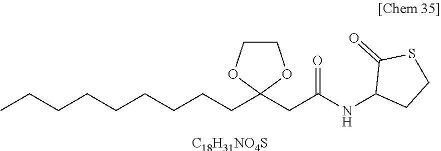 [SMILES: CCCCCCCCCC1(CC(=O)NC2CCSC2=O)OCCO1 ; "C18H31NO4S"] [0251] Prepared by modified GP5 with a yield of 53%. ¹H RMN (300 MHz, CDCl₃): δ6.89 (d, J=6.6 Hz, 1H, NH), 4.56 (dt, J=13.1, 6.7 Hz, 1H, C_{α}H), 4.10 to 3.93 (m, 4H, OCH₂CH₂O), 03:34 (td, J=11.7, 5.1 Hz, 1H, H_{B}), 3.22 (ddd, J=11.4, 7.1, 1.3 Hz, 1H, H_{A}), 2.86 (dddd, J=12.1, 6.7, 5.1, 1.4 Hz, 1H, H_{C}), 2.61 (s, 2H, C (2) H₂), 1.92 (dq, J=12.4, 7.0 Hz, 1H, H_{D}), 1.70-1.62 (m, 2H, C (4) H₂), 1:38-1:32 (m, 2H, C (5) H₂), 1.23 (s, 12H, C (6) H₂ to C (11) H₂), from 0.90 to 0.81 (m, 3H, C (12) H₃). ¹³C RMN (75 MHz, CDC₃): δ 205.32 (C═O), 169.79 (C₁, 109.75 (C₃), 65.17 (OCH₂CH₂O), 65.03 (OCH₂CH₂O), 59.27 (CH), 44.36 (C₂), 37.59, 31.96, 31.92, 29.77, 29.60, 29.59, 29.39, 27.59, 23.79, 22.76, 14:21 (C₁₂). R_{f} (1:1 EtOAc/Cychex): 0.31. HRMS (ESI): exact mass calculated for C₁₈H₃₁NO₄SH ([M+H]⁺): 358.2047. Found: 358.2047.

### 2-Nonyl-N-(3(S)-tetrahydro-2-oxo-3-thienyl)-1,3-dioxalane-2-acetamide 7g[429675-24-1]

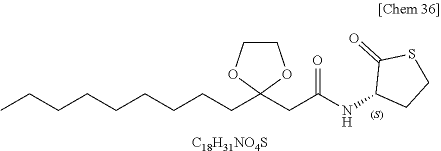 [0253] Prepared by GP5bis with a yield of 64%. ¹H RMN (300 MHz, CDCl₃): δ 6.89 (d, J=6.6 Hz, 1H, NH), 4.56 (dt, J=13.1, 6.7 Hz, 1H, H), 4.10 to 3.93 (m, 4H, OCH₂CH₂O), 03:34 (td, J=11.7, 5.1 Hz, 1H, H_{B}), 3.22 (ddd, J=11.4, 7.1, 1.3 Hz, 1H, H_{A}), 2.86 (dddd, J=12.1, 6.7, 5.1, 1.4 Hz, 1H, H_{C}), 2.61 (s, 2H, C (2) H₂), 1.92 (dq, J=12.4, 7.0 Hz, 1H, H_{D}), 1.70 to 1.62 (m, 2H, C (4) H₂), 1.38-1.32 (m, 2H, C (5) H₂), 1.23 (s, 12H, C (6) H₂ to C (11) H₂), 0.90 to 0.81 (m, 3H, C (12) H₃). ¹³C RMN (75 MHz, CDCl₃): δ205.32 (C═O), 169.79 (C), 109.75 (C₃), 65.17 (OCH₂CH₂O), 65.03 (OCH₂CH₂O), 59.27 (CH), 44.36 (C₂), 37.59, 31.96, 31.92, 29.77, 29.60, 29.59, 29.39, 27.59, 23.79, 22.76, 14:21 (C₁₂). R_{f} (1:1 EtOAc/Cychex): 0.31. HRMS (ESI): exact mass calculated for C₁₈H₃₁NO₄SH ([M+HA]⁺): 358.2047. Found: 358.2047.

### N-((1S,2S)-2-hydroxycyclohexyl)-2-(2-nonyl-1,3-dioxalan-2-yl) acetamide 7h

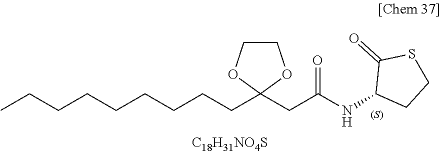 [0255] Prepared by GP5 with a yield of 54%. ¹H RMN (300 MHz, CDCl₃): δ6.47 (d, J=7.5 Hz, 1H, N H), 3.96 (p, J=3.7, 3.1 Hz, 4H), 3.66-3.53 (m, 1H, NHCHC(OH) H), 3.28 (td, J=9.9, 4.3 Hz, 1H, NHC H), 2.63-2.49 (m, 2H, C (2) H₂), 2.01 (ddd, J=11.9, 4.9, 2.5 Hz, 1H, NHCHC(H) H_{eq.}), 1.89 (dq, J=14.3, 4.6, 3.3 Hz, 1H, NHCHC(H) Hₐₓ), 1.73-1.59 (m, 4H, C (4) H₂ and NHCHCH(OH) CH₂), 1.35-1.27 (m, 2H, C (5) H₂), 1.21 (s, 16H, C (6) H₂ to C (11) H₂ and NHCHCH₂CH₂CH₂CH₂C(OH) H), 0.83 (t, J=6.7 Hz, 3H, C (12) H₃). ¹³C RMN (75 MHz, CDCl₃): δ170.95 (C₁), 109.89 (C₃), 75.31 (CHOH), 65.04 (ketal), 65.02 (ketal), 55.45 (NHCH), 44.56 (C₂), 37.51, 34.35, 31.92 (C₄), 31.44 (C₅), 29.73, 29.58, 29.55, 29.35, 24.62, 24.62, 24.06, 23.71, 22.72, 14.16 (C₁₂). R_{f} (2:1 EtOAc/Cychex)=0.15. MS (ESI): exact mass calculated for C₂₀H₃₇NO₄Na ([M+Na]⁺): 355.52. Found: 378.1.

### N-((1S,2R)-2-hydroxycyclohexyl)-2-(2-nonyl-1,3-dioxolan-2-yl) acetamide 7i

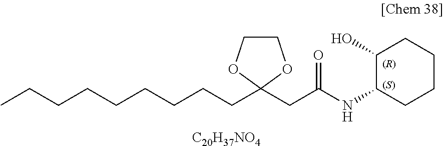 [0257] Prepared by GP5 with a yield of 51%. ¹H RMN (300 MHz, CDCl₃): δ6.70 (d, J=8.1 Hz, 1H, N H), 4:01-3,96 (m, 4H, ketal), 3.95-3.87 (m, 2H, NHCHC(OH) H and NHC H), 2.56 (d, J=2.7 Hz, 2H, C (2) H₂), 2:37 (s, 1H), 1.71-1.57 (m, 7H), 01:38 (ddd, J=14.3, 6.9, 4.0 Hz, 4H), 1.24 (d, J=2.2 Hz, 12H, C (6) H₂ to C (11) H₂), from 0.89 to 0.83 (m, 3H, C (12) H₃). ¹³C RMN (75 MHz, CDCl₃): δ169.32 (C₁), 110.03 (C₃), 69.52 (CHOH), 65.09 (ketal), 50.78 (NHCH), 44.75 (C₂), 37.52, 31.99, 31.53, 29.83, 29.64, 29.43, 27.37, 23.80, 23.48, 22.79, 20.45, 14.23 (C₁₂). R_{f} (3:1 EtOAc/Cychex)=0.26. HRMS (ESI): exact mass calculated for C₂₀H₃₇NO₄Na ([M+Na]⁺): 378.2615. Found: 378.2615.

### N-(5-chloro-2-hydroxyphenyl)-2-(2-nonyl-1,3-dioxolan-2-yl) acetamide 7m

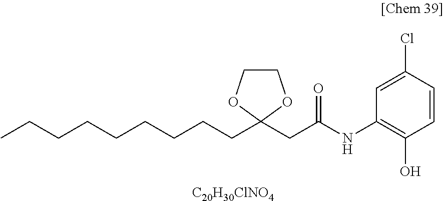 [SMILES: CCCCCCCCCC1(CC(=O)Nc2cc(Cl)ccc2O)OCCO1 ; "C20H30ClNO4"] [0259] Prepared by GP5 with a yield of 37%. ¹H RMN (300 MHz, CDCl₃): δ 8.78 (s, 1H, OH), 8.67 (s, 1H, NH), 7.07 (dd, J=8.7, 12.5 Hz, 1H, aromatic), 6.98 (d, J=2.5 Hz, 1H, aromatic), 6.94 (d, J=8.7 Hz, 1H, aromatic), 4.07 (s, 4H, OCH₂CH₂O), 2.80 (s, 2H, C (2) H₂), 1.75-1.69 (m, 2H, C (4) H₂), 1.43-1.36 (m, 2H, C (5) H₂), 1.28-1.25 (m, 12H, C (6) H₂ to C (11) H₂), 0.90-0.85 (m, 3H, C (12) H₃). ¹³C RMN (75 MHz, CDCl₃): δ169.75 (C₁), 147.68 (C OH), 127.04 (NH C), 126.73 (CCl), 124.94 (aromatic), 121.94 (aromatic), 121.17 (aromatic) 109.77 (C₃), 65.29 (acetal), 44.67 (C₂), 37.65, 32.01, 29.73, 29.62, 29.42, 23.80, 22.81, 14.25 (C₁₂). R_{f}(1:4 EtOAc/Cychex): 0.14. HRMS (ESI): exact mass calculated for C₂₀H₃₀ClNO₄H ([M+H⁺]): 384.1936. Found: 384.1936.

Procedure for the Synthesis of the Terminal Azido-Carboxylic Acid
The appropriate bromocarboxylic acid (1.0 equivalent) and the sodium azide (1.5 equivalents) were dissolved in DMF (5 ml/mmol carboxylic acid). The reaction mixture was heated to 60° C. and stirred overnight under argon atmosphere. After completion, the solvent was removed under reduced pressure. The resulting oil was dissolved in 1:1:1 (v/v/v) mixture of EtOAc, H₂O and brine. The organic phases are extracted with EtOAc (×3) and washed with semi-saturated brine (×2) before drying over MgSO₄. The solvents were removed under vacuum to give the product as an oil.

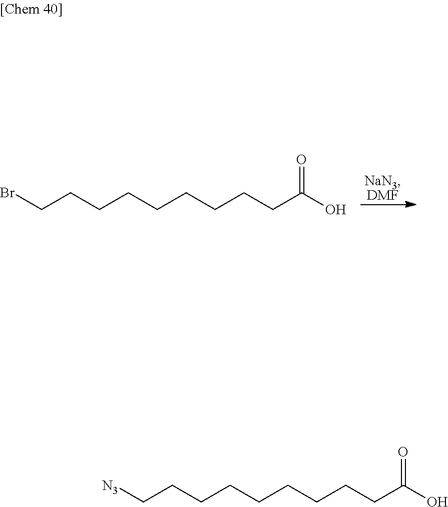

### Acid 10-Azidodecanoic 14 [186788-32-9]

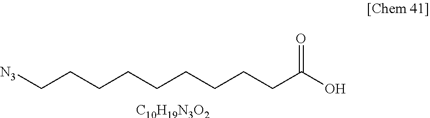 [0263] Prepared with a quantitative yield. ¹H RMN (300 MHz, CDCl₃): δ 3.24 (t, J=6.9 Hz, 2H, C (10) H₂), 2:33 (t, J=7.5 Hz, 2H, C (2) H₂), 1.60 (dt, J=10.1, 6.9 Hz, 4H, C (3) H₂ and C (9) H₂), 1.30 (m, 10H, C (4) H₂ to C (8) H₂). ¹³C RMN (75 MHz, CDCl₃): δ180.31 (acid), 51.59 (C₁₀), 34.18 (C₂), 29.36, 29.23, 29.19, 29.11, 28.94, 26.80, 24.76.

General Procedure for Removing the Acetal Protecting Group (GP6)
The AHL precursor or a protected analogue (1.0 equiv.) was dissolved in TFA (4 ml/mmol substrate) and water (1 ml/mmol substrate). DCM could be added if necessary to improve the solubility (up to 10 ml/mmol substrate). The reaction mixture was stirred at room temperature under argon atmosphere until a TLC analysis in EtOAc/CycHex indicate a complete consumption of the starting reagent (overnight). The reaction was stopped by adding a saturated NaHCO₃ solution and NaHCO3₍ₛ₎ until the pH was stabilized at 4-5, and the organic phase was extracted with DCM (×3). The combined organic phases were dried over MgSO₄ and the solvent removed under vacuum. If necessary, the product could be purified by flash chromatography on a silica column.

### N-(3(S)-oxododecanoyl) homoserine lactone 1 (=15a) [168982-69-2]

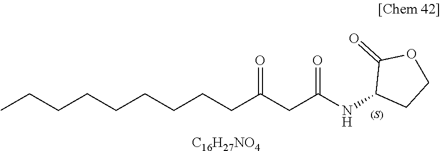 [0267] Prepared by GP6 with a yield of 98%. ¹H RMN (300 MHz, CDCl₃): δ 7.68 (d, J=6.6 Hz, 1H, N H), 4.59 (ddd, J=11.5, 8.7, 6.6 Hz, 1H, C_{α}H), 4.48 (dt, J=9.1, 1.4 Hz, 1H, H_{A}), 4.27 (ddd, J=11.1, 9.1, 6.0 Hz, 1H, H_{B}), 3.47 (s, 2H, C (2) H₂), 2.76 (dddd, J=12.6, 8.8, 6.0, 1.4 Hz, 1H, H_{C}), 2.52 (t, J=7.3 Hz, 2H, C (4) H₂), 2:30-2:15 (m, 1H, HD), 01.57 (d, J=6.9 Hz, 2H, C (5) H₂), 1.26 (t, J=3.1 Hz, 12H, C (6) H₂ to C (11) H₂), 0.90-0.85 (m, 3H, C (12) H₃). ¹³C RMN (75 MHz, CDCl₃): δ206.77 (C₃), 174.85 (ester), 166.44 (C₁), 65.99 (CH_{A}H_{B}), 49.19 (C_{α}), 48.12, 44.12, 31.99, 30.04, 29.52, 29.47, 29.38, 29.13, 23.51, 22.80, 14.24 (C₁₂). R_{f}(2:1 EtOAc/Cychex): 0.35. HRMS (ESI): exact mass calculated for C₁₆H₂₇NO₄Na ([M+Na]⁺): 320.1832. Found: 320.1834.

### 12-azido-3-oxo N-((3S)-tetrahydro-2-oxo-3-furanyl) dodecanamide 15b [1175052-13-7]

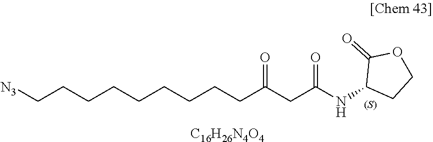 [0269] Prepared by GP6 with a yield of 87%. ¹H RMN (300 MHz, CDCl₃): δ 7.61 (d, J=4.9 Hz, 1H, N H), 4.58 (ddd, J=11.5, 8.7, 6.5 Hz, 1H, C_{α}H), 4.47 (dt, J=9.1, 1.5 Hz, 1H, H_{A}), 4.27 (ddd, J=11.0, 9.3, 6.1 Hz, 1H, H_{B}), 3.46 (s, 2H, C (2) H₂), 3.25 (t, J=6.9 Hz, 2H, C (12) H₂), 2.76 (dddd, J=12.6, 8.7, 6.0, 1.5 Hz, 1H, H_{D}), 2.52 (t, J=7.3 Hz, 2H, C (4) H₂), 2.22 (dtd, J=12.5, 11.2, 8.9 Hz, 1H, H_{C}), 1.59 (t, J=6.6 Hz, 2H, C (5) H₂), 1:37-1:27 (m, 12H, C (6) H₂ to C (11) H₂). ¹³C RMN (75 MHz, CDCl₃): δ 210.56 (C₃), 168.45 (ester), 165.97 (C₁), 65.99 (CH_{A}H_{B}), 51.61 (C₁₂), 49.21, 48.19, 44.06, 30.06 (CH_{C}H_{D}), 29.35, 29.33, 29.18, 29.06, 28.96, 26.81, 23.45. HRMS (ESI): exact mass calculated for C₁₆H₂₆N₄O₄Na ([M+Na]⁺): 361.1846. Found: 361.1848.

### 3-oxo-N-(tetrahydro-2-oxo-3-thienyl)-dodecanamide 15f [663883-93-0]

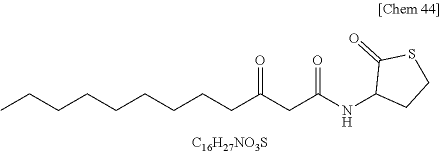 [SMILES: CCCCCCCCCC(=O)CC(=O)NC1CCSC1=O ; "C16H27NO3S"] [0271] Prepared by GP6 with quantitative yield. ¹H RMN (300 MHz, CDCl₃): δ7.47 (s, 1H, N H), 4.58 (dt, J=13.2, 6.8 Hz, 1H, C_{α}H), 3.45 (s, 2H, C (2) H₂), 3.40-3.22 (m, 2H, CH_{A}H_{B}), 2.86 (dddd, J=12.2, 6.7, 5.1, 1.5 Hz, 1H, H_{D}), 2.52 (t, J=7.4 Hz, 2H, C (4) H₂), 2.01 (dq, J=12.4, 7.1 Hz, 1H, H_{C}) 1.62-1.55 (m, 2H, C (5) H₂), 1.26 (t, J=2.8 Hz, 12H, C (6) H₂ to C (11) H₂), 0.91-0.84 (m, 3H, C (12) H₃). ¹³C RMN (75 MHz, CDCl₃): δ 206.76 (C₃), 204.67 (thioester), 166.39 (C₁), 59.40 (C_{α}), 48.34, 44.09, 31.99, 31.66, 29.52, 29.47, 29.38, 29.13, 27.62, 23.50, 22.80, 14.24 (C₁₂). HRMS (ESI): exact mass calculated for C₁₆H₂₇NO₃Na ([M+Na]⁺): 336.1604. Found 336.1605.

### 3-oxo N-[(3S)-tetrahydro-2-oxo-3-thienyl]-dodecanamide 15g [177158-29-1]

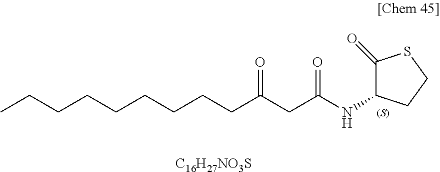 [0273] Prepared by GP6 with quantitative yield. ¹H RMN (300 MHz, CDCl₃): δ7.48 (d, J=6.6 Hz, 1H, N H), 4.58 (dt, J=13.3, 6.6 Hz, 1H, C_{α}H), 3.45 (s, 2H, C (2) H₂), 3.35 (td, J=11.5, 5.1 Hz, 1H, CH_{A}), 3.25 (ddd, J=11.5, 7.1, 1.5 Hz, 1H, CH_{B}), 2.84 (dddd, J=12.4, 6.7, 5.1, 1.5 Hz, 1H, H_{D}), 2.52 (t, J=7.3 Hz, 2H, C (4) H₂), 2.01 (dq, J=12.4, 7.1 Hz, 1H, H_{C}) 1.57 (t, J=7.3 Hz, 2H, C (5) H₂), 1.25 (t, J=3.1 Hz, 12H, C (6) H₂ to C (11) H₂), 0.91-0.82 (m, 3H, C (12) H₃). ¹³C RMN (75 MHz, CDCl₃): δ206.72 (C₃), 204.71 (thioester), 166.36 (C₁), 59.36 (C_{α}), 48.41 (CS), 44.06 (C₂), 31.98, 31.64, 29.51, 29.47, 29.37, 29.12, 27.61, 23.48, 22.79, 14.24 (C₁₂). HRMS (ESI): exact mass calculated for C₁₆H₂₇NO₃SH ([M+H]⁺): 314.1784. Found: 314.1785.

### N-[(1S,2S)-2-hydroxycyclohexyl]-3-oxo-dodecanamide 15h [886755-19-7]

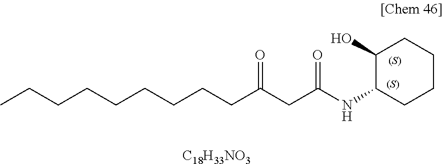 [0275] Prepared by GP6 with a yield of 54%. ¹H RMN (300 MHz, CDCl₃): δ7.17 (d, J=7.4 Hz, 1H, N H), 3.65 (dddd, J=11.2, 9.1, 7.4, 4.3 Hz, 1H, NHCHC(OH) Hₐₓ), 3.41 (s, 2H, C (2) H₂), 3:38-3:27 (m, 1H, NHC H), 2.51 (t, J=7.3 Hz, 2H, C (4) H₂), 2.9 to 1.99 (m, 1H, NHCHC(H) H_{eq}), 1.95 (tdd, J=7.4, 3.9, 2.3 Hz, 1H, NHCHC(H) Hₐₓ), 1.71 (ddt, J=8.8, 5.6, 2.7 Hz, 2H NHCHCH(OH) CH₂), 1.56 (p, J=6.9 Hz, 2H, C (5) H₂), 1.36-1.16 (m, 16H, C (6) H₂ to C (11) H₂ and NHCHCH₂CH₂CH₂CH₂C(OH) H, 0.90-0.81 (m, 3H, C (12) H₃). ¹³C RMN (75 MHz, CDCl₃): δ207.55 (C₃), 167.37 (C₁), 75.24 (CHOH), 55.88 (NHCH), 48.60, 44.08 (C₂), 34.39, 31.97, 31.38, 29.50, 29.46, 29.36, 29.11, 24.67, 24.10, 23.48, 22.78, 14.22 (C₁₂). HRMS (ESI): exact mass calculated for C₁₈H₃₃NO₃Na ([M+Na]⁺): 334.2353. Found 334.2353.

### N-[(1S,2R)-2-hydroxycyclohexyl]-3-oxo-dodecanamide 15i [897031-37-7]

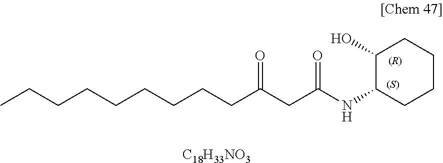 [0277] Prepared by GP6 with a yield of 67%. ¹H RMN (300 MHz, CDCl₃): δ7.25-7.14 (m, 1H, N H), 3.98 (ddd, J=8.2, 6.2, 2.7 Hz, 1H, NHC H), 3.92 (dt, J=5.8, 2.6 Hz, 1H, NHCHC(OH) H_{eq}), 3.40 (d, J=1.5 Hz, 2H, C (2) H₂), 2.52 (t, J=7.3 Hz, 2H, C (4) H₂), 2.20-2.08 (m, 2H), 1.68-1.53 (m, 6H), 1.42 (ttd, J=11.5, 5.5, 5.0, 2.8 Hz, 2H), 1.26 (d, J=3.5 Hz, 12H, C(6)H₂ to C(11)H₂), 0.91-0.83 (m, 3H, C (12) H₃). ¹³C RMN (75 MHz, CDCl₃): δ 207.31 (C₃), 165.84 (C₁), 69.38 (COH), 51.23 (NHCH), 49.17, 44.07, 31.99, 31.51, 29.52, 29.48, 29.38, 29.15, 27.32, 23.52, 23.44, 22.79, 20.43, 14.23 (C₁₂). R_{f} (3:1 EtOAc/CycHex)=0.38. HRMS (ESI): exact mass calculated for C₁₈H₃₃NO₃H ([M+H]⁺): 312.2533. Found: 312.2534.

### N-(5-chloro-2-hydroxyphenyl)-3-oxododecanamide 15 m [663883-68-9]

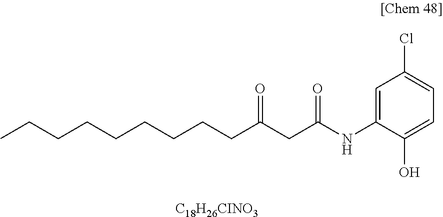 [SMILES: CCCCCCCCCC(=O)CC(=O)Nc1cc(Cl)ccc1O ; "C18H26CINO3"] [0279] Prepared by GP6 with a yield of 98%. ¹H RMN (300 MHz, MeOD-d₄): δ8:01 (d, J=2.5 Hz, 1H, aromatic H), 6.94 (dd, J=8.6, 2.6 Hz, 1H, aromatic H), 6.84 to 6.76 (m, 1H, aromatic H), 3.64 (dd, J=5.5, 3.3 Hz, 1H, C (2) H-form-enol), 2.60 (t, J=7.3 Hz, 2H, C (4) H₂), 1.59 (p, J=7.2 Hz, 2H, C (5) H₂), 1.34-1.26 (m, 12H, C (6) H₂ to C (11) H₂), 0.92 to 0.87 (m, 3H, C (12) H₃). ¹³C RMN (75 MHz, MeOD-d₄): δ 207.41 (C₃), 167.64 (C₁), 147.59 (COH), 128.30 (aromatic C), 125.51 (aromatic C H), 124.91 (aromatic C), 122.45 (aromatic C H), 116.95 (aromatic C H), 43.99 (C₂), 33.03 (C₄), 30.57, 30.53, 30.40, 30.13, 24.45, 23.72, 14.43 (C₁₂). HRMS (ESI): exact mass calculated for C₁₈H₂₆ClNO₃H ([M+H]⁺): 340.1654. Found 340.1663.

3. Anti-Inflammatory Effect of the Compounds of the Invention

In order to evaluate the properties of compounds of interest, a murine macrophage line, the RAW264.7, was used.

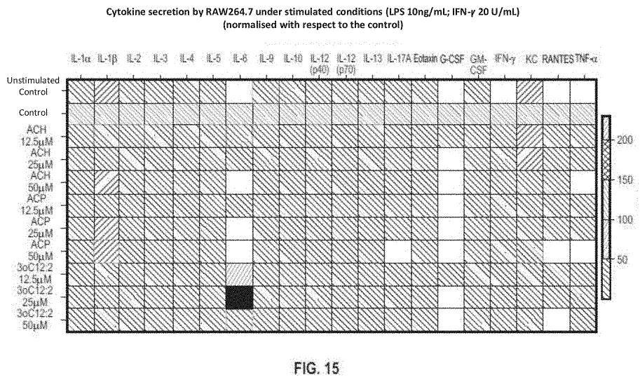

To assess the effect on the inflammation, the cells were treated or not with the addition of a proinflammatory cocktail (interferon-γ (IFN-γ, 20 U/mL) and lipopolysaccharide (LPS, 10 ng/mL)). The inflammatory state was assessed by Multiplex analysis by dosing the secretion of 23 cytokines in the supernatant of the cells. The heatmap results are shown in FIG. 15, which shows the secretion of the cytokines by RAW264.7 under stimulated conditions (LPS 10 ng/mL; IFN-γ 20 U/mL) (normalized in relation to the control).

These results allow to visualize globally a decrease of the production of the cytokines in the presence of 50 μM PCA. The cytokines whose production was modulated by the compounds of the invention are interleukin-13 (IL-1p), IL-2, IL-6, IL-12, RANTES, TNFα, pro-inflammatory cytokines. The decrease in these proteins was dose-dependent, and the largest effects were observed in the presence of 50 μM PCA.

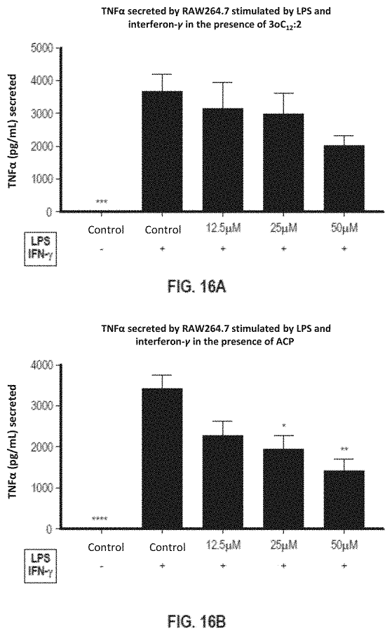

To illustrate this, 2 histograms (the first with the 3oxoC₁₂:2; the 2^{nd} with the PCA) showing the results observed for the TNF alpha (results from the Multiplex analysis) are shown in FIGS. 16A and 16B (at the top: TNF∝ secreted by RAW264.7 stimulated by LPS and interferon-γ in the presence of 3oC₁₂:2 and at the bottom: TNF∝ secreted by RAW264.7 stimulated by LPS and interferonγ—in the presence of PCA).

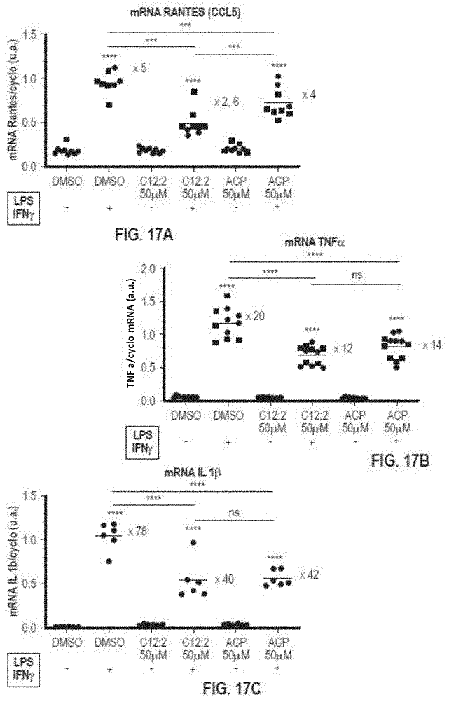

The confirmation of some gene expression results was performed by measuring the messenger RNA by quantitative PCR, in particular for 3 cytokines of interest: Rantes, TNF aplha, IL1-beta. The results are shown in the diagrams shown in FIGS. 17A-17C.

Thus, we observe an anti-inflammatory effect of the compounds on the cells of the macrophage type both at the protein level and in mRNA expression.

4 Measurement of the Toxicity of the Compounds of the Invention

4.1 Measurement of the Toxicity of the Compounds on Eukaryotic Cells

The cytotoxicity of the tested and control compounds was evaluated using a measurement test of the LDH (Lactate Dehydrogenase) secretion. This test is based on the measurement of the amount of LDH secreted by the cells in their supernatant, compared to the amount of LDH that will remain in the intracellular compartment. This ratio provides an indication of the membrane damage suffered by the cells, and thus of the cytotoxicity of the compound. A compound is considered toxic when the ratio is higher than 10%.

Two methods were used to perform this test: the measurement using an extemporaneously prepared pyruvate/NADH solution, or using the Cytotoxicity Detection Kit^{PLUS}(LDH) from the manufacturer Roche (Sigma-Aldrich).

Pyruvate/NADH method: pyruvate/NADH solution prepared with 4.1 mg pyruvic acid (0.62 mM) and 7.7 mg NADH (0.18 mM) in 60 ml of 0.1M PBS (pH 7.4).

For the measurement of the LDH concentration in the supernatants, 800 μL of NADH was added to 200 μL of supernatant in plastic spectrometry cuvettes, and the decrease in absorbance of the final solution was read at 340 nm for 1 min. For the measurement of the LDH concentration in the cell lysates, 800 μL of NADH was added to 10 μL of cell lysates and 190 μL of 0.1M PBS in plastic spectrometry cuvettes, and the decrease in absorbance of the final solution was read at 340 nm for 1 min. The percentage of LDH secreted was then calculated by the ratio of the decay slopes of the absorbance of the supernatants and of the cell lysates (respectively).

Cytotoxicity Detection Kit^{PLUS} (LDH) method: absorbance test performed according to the manufacturer's instructions. The percentage of LDH secreted was then calculated by the formula:

$${\%}_{LDH} = \frac{{DO}_{sample} - {DO}_{basal control}}{{DO}_{control activated} - {DO}_{basal control}} * 100 \qquad [ Math 2 ]$$

4.2 Method for Measuring the Toxicity of the Compounds on Bacterial Line

The E. coli K12 strain was cultured at D0 on agar gel, and then a selected colony was transferred to a liquid bacterial culture LYBHI medium at D1. At day 2, this colony was diluted 1:100 in LYBHI medium, and maintained for 18 hours for expansion before distribution in an opaque 96-well plate. In each well were distributed: LYBHI medium, bacterial culture, and test or control compounds. The absorbance of the wells was read at 600 nm at t0 and t18h. The raw absorbance values were corrected against the absorbance of the wells containing only LYBHI medium without bacteria or compounds.

4.3 Results on Eukaryotic Cells

a. Natural AHL 3oxoC₁₂-HSL and 3oxoC₁₂:2-HSL

FIGS. 18A-18B show that in the Caco-2/TC7 cell line, the 2 molecules are well tolerated in the concentration range 1-100 μM, in the presence and absence of pro-inflammatory cytokines:

the measured cytotoxicity does not exceed 2.5% for 3oxoC₁₂-HSL and 1.5% for 3oxoC₁₂:2-HSL. In comparison, the toxicity of the activated control (DMSO 0.1% and cytokines) is about 4%.

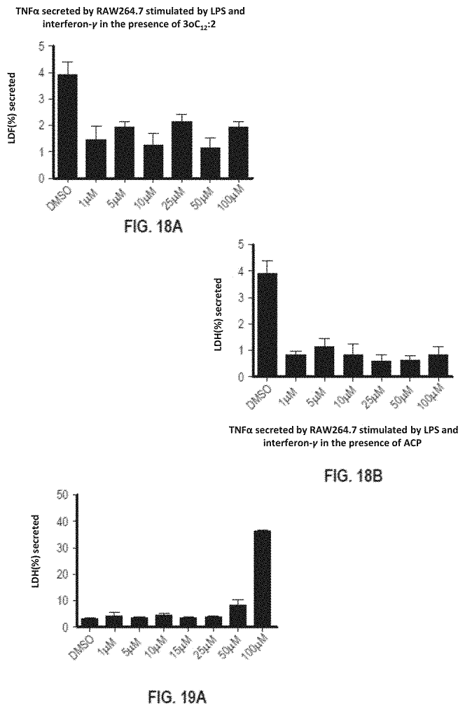

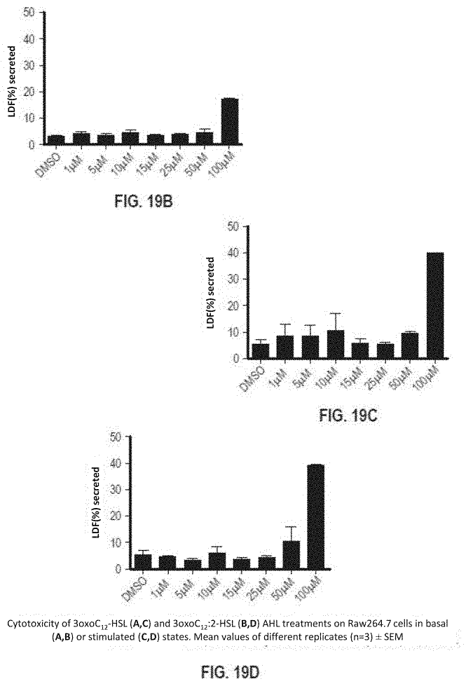

FIGS. 19A-19D show that in the Raw264.7 murine cell line, an increase in secreted LDH was observed as early as 50 μM for both AHL, with a cytotoxicity greater than 10% at the 100 μM concentration. This phenomenon is observed in basal and stimulated conditions, and is therefore attributable to a toxicity of the molecules. This justifies the use of the 1-50 μM doses in the rest of the study on the macrophage line.

b. (D/L)-3oxoC₁₂-HTL (Formula I-1) and (S)-3oxoC₁₂-HTL (Formula I-3)

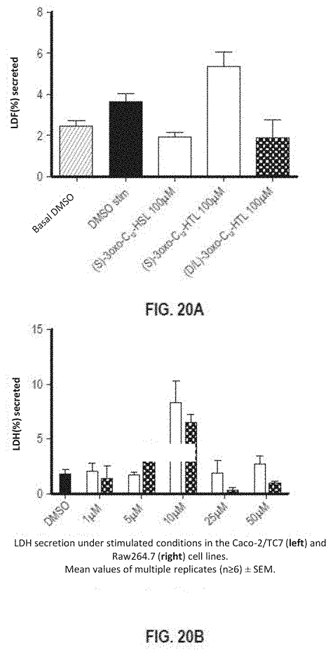

As can be seen in FIGS. 20A-20B, neither of these two thiolactone-headed compounds showed toxicity on the two cell lines, with LDH secretions below 10% even at the highest concentrations.

c. (S,S)-3oxoC₁₂-ACH (Formula (I-2) and (R,S)-3oxoC₁₂-ACH (Formula (I-4)

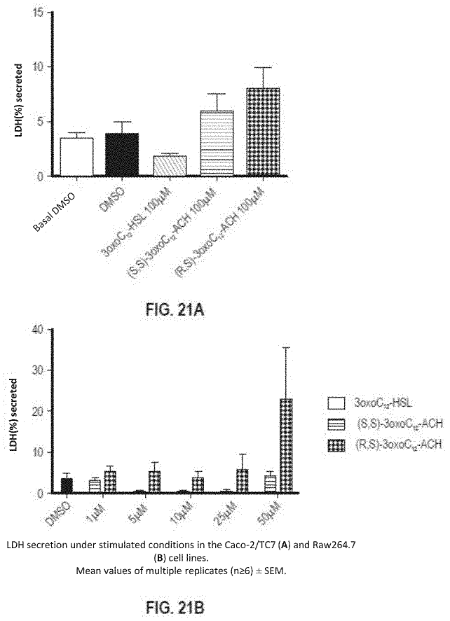

Both compounds are not cytotoxic at low concentrations as shown in FIGS. 21A-21B, but the (R,S)-3oxoC₁₂-ACH molecule shows an increase in LDH secretion at the higher concentrations (≥50 μM). On the Caco-2/TC7 epithelium cells, without exceeding the 10% mark, we observe a secretion of 9% at 100 μM. This effect is not as significant for the (S,S)-3oxoC₁₂-ACH diastereomer.

This observation is found in the Raw264.7 macrophage line, where consistently, the LDH secreted in the presence of (R,S)-3oxoC₁₂-ACH is greater than that secreted in the presence of (S,S)-3oxoC₁₂-ACH, at all concentrations. The (R,S)-3oxoC₁₂-ACH molecule is also toxic at 50 μM.

d. 3oxoC₁₂-Aminochlorophenol (Formula I-6)

The molecule is not toxic to either line at any of the concentrations tested as shown in FIGS. 22A-22B.

4.4 Results on Bacterial Strain

a. Natural AHL 3oxoC₁₂-HSL and 3oxoC12:2-HSL

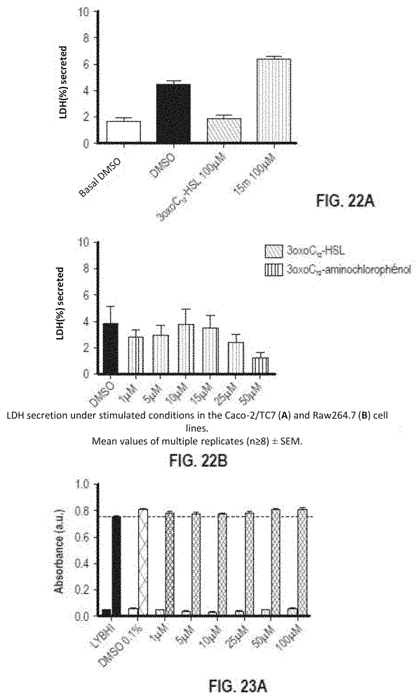

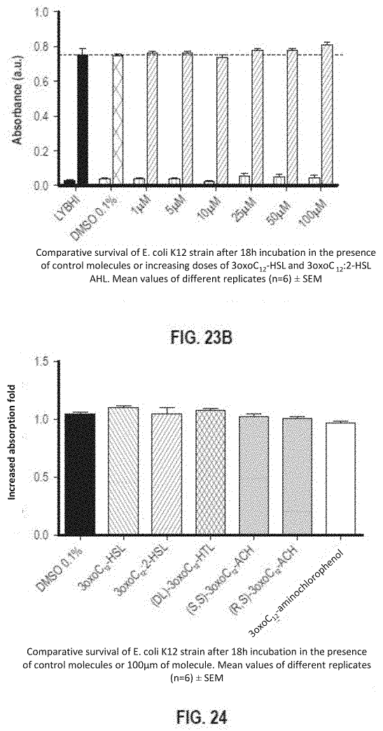

The 3oxoC₁₂-HSL and 3oxoC₁₂:2-HSL AHL were tested for bactericidal effects in the 1-100 μM range. No toxic effect was observed after 18 h of incubation: the absorbances recorded were identical to those for the LYBHI medium alone and in the presence of 0.1% DMOS: see FIGS. 23A-23B.

4.5 Results on Bacterial Strain

a. (D/L)-3oxoC12-HTL (Formula I-1), (S)-3oxoC12-HTL (Formula I-3), (S,S)-3oxoC12-ACH (Formula I-2), (R,S)-3oxoC12-ACH (Formula I-4) and 3oxoC12-Aminochlorophenol (Formula I-6) Analogues

No significant differences were observed among all the concentrations and all the molecules tested, so only the maximum concentration (100 μM) is shown in FIG. 24.

In general, no compound, natural or synthetic, is bactericidal on the E. coli K12 strain.

1. A compound having the following general formula I: 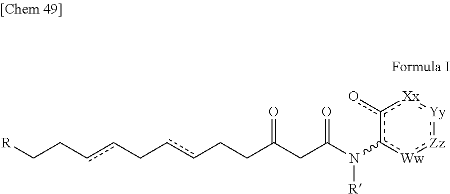
wherein:
X, Y, Z and W are independently of each other a carbon atom or a heteroatom selected from S, N and O, provided that X is different from O,
X, Y, Z and W are independently of each other optionally substituted with a halogen selected from Cl, F, Br, and I, or a linear or branched C₁ to C₄ alkyl group,
x, y, z, and w, independently of each other, are 0 or 1, provided that 3≤x+y+z+w≤4,
R represents H or a linear or branched C₁ to C₄ alkyl group, or a hydroxyl group (OH) or an azido group (N₃), [symbol]
represents a single or double bond (cis or trans),
R′ represents H or a linear or branched C₁ to C₄ alkyl group for use in the treatment of an inflammatory disease of the epithelium.

2. The compound according to claim 1 selected from the group consisting of:
the (D/L)-3-oxo-C₁₂ aminothiolactone ((D/L)-3-oxo-C₁₂-HTL) of the following formula I-1: 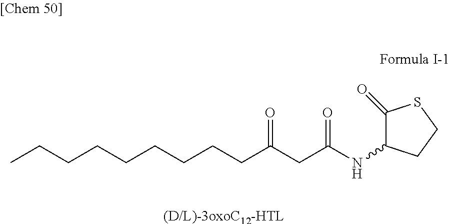 [SMILES: CCCCCCCCCC(=O)CC(=O)NC1CCSC1=O ; "(D/L)-3oxoC12-HTL"]
the (S,S)-3-oxo-C₁₂ aminocyclohexanol ((S,S)-3-oxo C₁₂-ACH) of the following formula I-2: 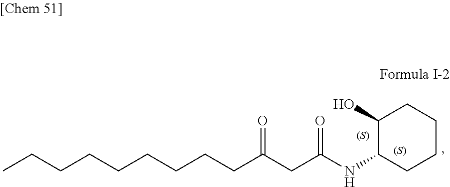
the (S)-3-oxo-C₁₂ aminothiolactone (S)-3-oxo-C₁₂-HTL) of the following formula I-3: 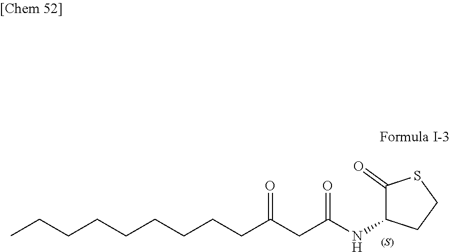
the (R,S)-3-oxo-C12-aminocyclohexanol of the following formula I-4: 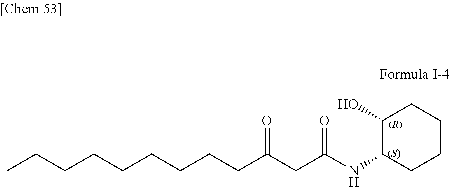
the 3-oxo-C12-aminocyclohexanol of the following formula I-5: 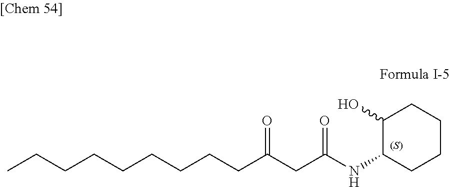
the 3-oxo-C12-aminochlorophenol of the following formula I-6:

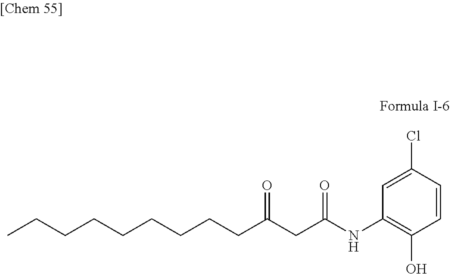 [SMILES: CCCCCCCCCC(=O)CC(=O)Nc1cc(Cl)ccc1O]

3. A method for the treatment of an inflammatory disease of the intestine comprising administering an effective amount of the compound of claim 1.

4. A method for the treatment of psoriasis comprising administering an effective amount of the compound of claim 1.

5. A pharmaceutical composition comprising at least one compound having the following general formula I: 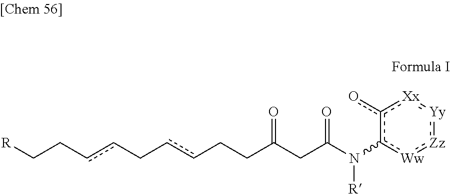
wherein:
X, Y, Z and W are independently of each other a carbon atom or a heteroatom selected from S, N and O, provided that X is different from O,
X, Y, Z and W are independently of each other optionally substituted with a halogen selected from Cl, F, Br, and I, or a linear or branched C₁ to C₄ alkyl group,
x, y, z, and w, independently of each other, are 0 or 1, provided that 3≤x+y+z+w≤4,
R represents H or a linear or branched C₁ to C₄ alkyl group, or a hydroxyl group (OH) or an azido group (N₃),
R′ represents H or a linear or branched C₁ to C₄ alkyl group, [symbol]
represents a single or double bond (cis or trans), and
at least one pharmaceutically acceptable excipient.

6. The pharmaceutical composition of claim 5 wherein the at least one compound of formula I is selected from the group consisting of:
the (D/L)-3-oxo-C12-aminothiolactone ((D/L)-3oxoC12-HTL) of the following formula I-1: 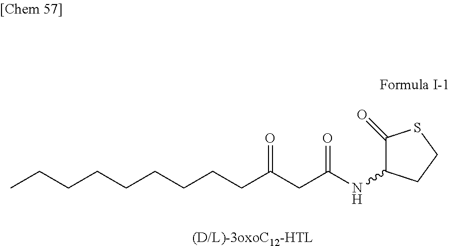 [SMILES: CCCCCCCCCC(=O)CC(=O)NC1CCSC1=O ; "(D/L)-3oxoC12-HTL"]
the (S,S)-3-oxo-C12-aminocyclohexanol (S,S) 3-oxo-C12-ACH) of the following formula I-2: 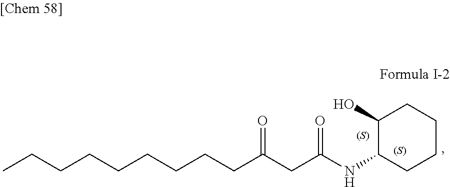
the (S)-3-oxo-C12-aminothiolactone ((S)-3-oxo-C12-HTL) of the following formula I-3: 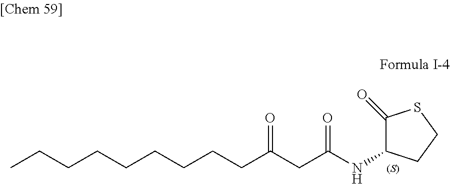
the (R,S)-3-oxo-C12-aminocyclohexanol of the following formula I-4: 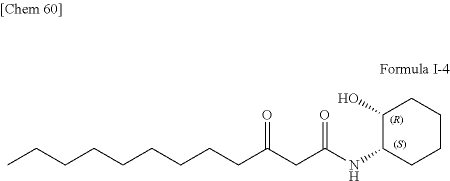
the 3-oxo-C12-aminocyclohexanol of the following formula I-5: 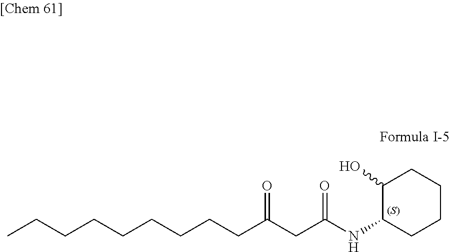
3-oxo C12-aminochlorophenol of the following formula I-6:

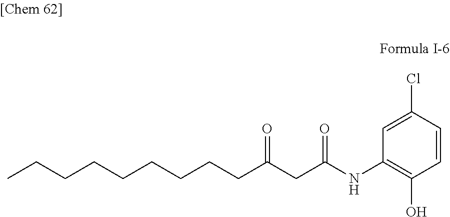 [SMILES: CCCCCCCCCC(=O)CC(=O)Nc1cc(Cl)ccc1O]

7. A method for the treatment of an inflammatory disease of the epithelium comprising administering an effective amount of the pharmaceutical composition of claim 5.

8. A method for the treatment of an inflammatory disease of the intestine comprising administering an effective amount of the pharmaceutical composition of claim 5.

9. A method for the treatment of psoriasis comprising administering an effective amount of the pharmaceutical composition of claim 5.
